SEQUENCE LISTING (32 sequences)

SEQ ID NO: 1 - DNA, 3531 bp, Arabidopsis thaliana
  source 1..3531: mol_type = unassigned DNA
atgtctataacttgtgagctcttgaatcttacttcaaagaaagctaagaagtcgtcgagcagtgacaagaaatggctaaagaagcctctcttcttcctgattttgtgtggctctttggtaattgttttggttatgttcttacggttaggtagaagtcagaaggaggagacagattcttgtaatggagaagagaaagtgttgtatagacatcaaaatgtcacaagaagtgagattcatgatttggtctctttgttctctgattcagatcaggtaacatcctttgaatgtcataaggaatcaagccctggaatgtggacaaactatggtattacatgttccctgagtgtgcgttctgataaacaagagactagagggcttccctggaatcttggcttaggacattctatctcatcaacatcttgtatgtgtggtaatcttgaaccgattttacagcaacctgaaaaccttgaggaagaaaaccatgaagaagggctggagcagggtttgtcatcgtatttaagaaatgcatggtggtgtctaatccttggtgtgttagtgtgccataagatttatgtatctcattctaaagcacgaggtgagaggaaagagaaagtacatctgcaagaggctttagctccaaagaagcagcaacaacgtgctcagacttcttctagaggggctggaagatggaggaagaatatccttctccttggtattttaggaggagtttccttctctgtttggtggttttgggacactaatgaggagatcataatgaaaaggagggagactttggcaaacatgtgtgacgaacgagcacgtgttttacaagatcagttcaatgttagcttgaaccatgttcatgccttgtctattcttgtatctacatttcatcatggtaaaatcccatctgccattgatcagagaacatttgaagaatatactgagagaacaaactttgagaggccacttactagtggtgtagcgtatgctttgaaagtcccacactcagaaagagagaaatttgaaaaggagcatggatgggcaataaagaaaatggaaactgaggaccagacagttgtacaagattgtgttcctgaaaactttgatcccgcaccgattcaagacgaatacgcgccagttatatttgctcaagaaactgtttcccatattgtatcggtcgacatgatgtctggagaagaagaccgtgaaaacatcttacgggcaagggcatcaggaaaaggagtgttaacatctccatttaagcttcttaagtcaaatcatcttggtgttgtgttgacctttgctgtctatgacacgagcctaccgcctgatgctacagaagaacagcgtgttgaagcaactattgggtaccttggtgcatcatatgatatgccatcgctggtggagaaacttcttcaccaacttgccagcaaacagacaattgctgtggatgtttacgacacaactaacacttcaggtctaataaaaatgtatggctcagaaattggggatataagtgagcagcatataagtagccttgattttggtgatccatcaaggaaccatgagatgcattgcaggtttaagcataaacttcccattccctggacagcgataacaccgtcgatcttagttctggttattacttttcttgttggttatattttatatgaagccatcaaccgaattgcgacagttgaagaggattgtcagaagatgagggaactcaaagctcgtgctgaggccgctgacattgcaaagtcacagttcctagcaactgtttctcatgagatacggactccgatgaatggagttttaggaatgctgaaaatgctgatggacaccgatcttgatgcgaagcagatggactatgcgcaaactgctcatggcagtgggaaggatcttacatcactaataaatgaggttcttgatcaggcaaagattgaatccggaaggctcgagcttgaaaatgtgccttttgatatgcgttttattcttgataatgtttcatctctcctctctggcaaggcaaatgaaaaaggaattgagttggccgtttatgtttctagtcaagttcctgatgttgtagtcggtgatccgagtcggttccggcagatcattacaaacctggttggaaactcaatcaaattcacacaggaaaggggacacatatttatctcagtgcaccttgcagatgaggtaaaggagcctcttactattgaagacgcagtgctaaaacagcgactagctttaggatgcagcgagtccggtgagacagttagcgggtttcctgcggtaaatgcatggggaagctggaagaatttcaagacatgttacagtactgagagtcagaattctgatcaaatcaaattgctagttacagtggaggacactggagttggcatacctgtggatgcacaaggccgaatcttcacaccttttatgcaagccgacagttccacatcgcggacttatggtggaactggcataggtttgagtataagcaaacgtttggttgaactcatgcaaggagagatggggtttgtgagtgagcccgggataggcagtactttttcatttactggagttttcgggaaagcagaaacaaatacgtcgattactaagctggaacgattcgatctagctattcaggagtttacaggattgagagcattagttattgataacagaaacattagagcagaggtcaccaggtacgaacttcggagactgggaatatctgcagacattgtttcaagtctgagaatggcatgcacttgttgtatcagcaaattagaaaatttggctatgattctaatagacaaagacgcctggaacaaggaagaattttcagtacttgacgagttgtttacccgaagcaaagtaacctttacaagagtcccaaagatttttcttttggcaacttctgcaactcttactgagcgcagtgagatgaagtctactggtctcatcgatgaggtggtgataaagcctcttcggatgagtgtcttaatatgttgcttgcaagaaacccttgtcaatggcaagaagaggcaaccgaacagacagcgaagaaatcttggacacttgctaagagaaaaacagattctggttgtggatgataatcttgtgaacagacgagttgcagaaggtgcacttaagaaatatggagctattgttacatgcgttgagagtggcaaagctgcattggcaatgcttaagccgcctcataacttcgatgcttgcttcatggatctccagatgcctgaaatggatggatttgaagcgacaaggagagtccgtgagctggagagggaaatcaataagaaaatagcttctggagaagtttcagctgaaatgttctgtaaatttagtagttggcacgtcccgatattagcaatgacagcagatgttattcaggctactcatgaagaatgcatgaaatgtggaatggatggttatgtatcaaaaccgtttgaagaggaagtgctctacacagcggtagcaagattctttgaaccttgttaa

SEQ ID NO: 2 - DNA, 3111 bp, Arabidopsis thaliana
  source 1..3111: mol_type = unassigned DNA
atgagtctgttccatgtgctagggtttggtgtcaagattgggcatctcttctggatgctatgctgctggtttgtttcttggttcgttgataatgggatcgaggacaagtctggtcttttagttggctctgtcggtgatcttgagaagactaagatgactacgttgaagaagaagaacaagatgtggttctggaataagatctctagcagcggactcaagatcccgagtttctcttatcagtttcttggctctgttaaattcaacaaggcgtggtggaggaagcttgtggtggtttgggttgtcttctgggtcttggtctctatttggacgttttggtactttagctcgcaagctatggagaagaggaaagagacgctagctagtatgtgtgatgagagagctcgtatgctgcaggatcagttcaacgttagcatgaatcatgttcaagccatgtctatcttgatctcaaccttccaccatggcaagattccttctgctatcgatcagagaacattctcagagtacactgatagaacttcctttgagaggcctcttactagcggggtagcttatgctatgagggtgctccattcagagagggaagagttcgagaggcaacaaggttggactattaggaagatgtattctcttgaacaaaacccagttcacaaggatgactatgacctggaagctttggaaccatcccctgtccaagaagagtacgctccagtcatctttgctcaggacactgtttctcacgttgtttctctcgatatgctgtctgggaaagaagatcgtgaaaacgttttgcgggccaggagttcaggtaaaggggttttgacagctcctttcccattgataaagacaaatagacttggggtgatcctgacatttgcagtgtacaagagagatctcccctccaatgcaacgccaaaagagagaattgaggctactaacgggtatctcgggggagtgtttgacattgagtccctggtagaaaacttgcttcaacagctggctagcaagcaaacgattcttgtcaatgtgtacgatatcaccaatcactctcaaccgattagcatgtatggtacaaatgtgtcggctgatgggttggaacgtgttagtccactaatctttggcgatccattgagaaagcatgagatgcgttgcagatttaagcagaaaccaccatggccagtgctatcaatggtgacatcattcggtatccttgtgattgcgttacttgttgcacatataatccacgcaaccgttagtcgaatacacaaagttgaagaagattgtgataaaatgaagcagctcaagaaaaaggctgaagcagcagatgttgcaaagtcacagttccttgccactgtttcacatgaaatcagaactccaatgaatggtgttctaggaatgttgcatatgcttatggacacagagttagatgttacgcaacaggattatgttaggaccgcacaggcaagtggaaaagctttagtctcgctaataaatgaggttttggaccaagcaaagattgaatctggaaagcttgaacttgaggaggtgcggtttgatttgagaggaatattagatgatgtcctgtcactcttctctagcaagtcccaacaaaagggggtggagttggcagtatacatatctgatcgtgttccagatatgttaattggtgatcctgggaggtttcgacaaatactcacaaatcttatgggtaattccattaagttcactgagaaaggacacatctttgtaactgttcatttggtggatgagctatttgaatctatcgatggagagacagcatcatctccggaaagtacactgagtgggcttccagttgcagaccggcagaggagctgggaaaactttaaagctttcagctccaacgggcatcggagctttgaaccatctccccctgatataaacctaatcgtctcagttgaggatactggcgtagggatccctgtagaagcgcagtcccgtatttttacgcctttcatgcaagtcggaccatccatatccaggacgcatggaggcacaggaattggacttagcataagcaaatgtctagttggactgatgaagggagaaattggattctcgagtactcccaaggttgggtccacattcacatttactgctgtattttccaatgggatgcaaccagctgaaagaaagaatgacaacaaccagcccatattctcggaattccggggcatgaaagctgtggttgtggaccataggcctgcaagggcaaaagtctcgtggtaccattttcagcgtcttggaattcgagtcgaagtagttccacgtgttgaacaggctctacattatctgaagattggtactaccactgtgaatatgatactcatagagcaagaaatatggaatagggaagcagatgatttcattaaaaagctacagaaagaccctcttttcctttctcctaagttgattttgttagcaaactcagtagaatcgtcaatatcagaggctttatgcaccggtatagatcctccaatagtgatagtgaaaccattgagggcgagtatgctagcagcaactttgcagaggggattgggtattggaatcagagaaccacctcaacacaagggacctcctgctttgattctcaggaatcttctccttggtagaaaaattttaatcgtggatgataacaacgtaaacctcagagtggcagcgggagctctgaaaaagtacggagctgatgtggtctgcgctgagagtgggataaaggcaatctcattgcttaagccacctcacgagtttgatgcttgcttcatggacattcagatgccagaaatggatggatttgaagctacaaggagaatacgagatatggaagaggagatgaacaagagaataaagaatggggaggctttgatagtagagaacggtaacaaaacaagctggcatcttccggtattagcaatgacggcagatgtgatccaagcaacgcatgaggaatgtctgaagtgtggaatggatgggtatgtatcaaaaccatttgaagcagagcagctgtacagggaagtttctcgctttttcaattcgccttcagatacagaatcataa

SEQ ID NO: 3 - DNA, 3243 bp, Arabidopsis thaliana
  source 1..3243: mol_type = unassigned DNA
atgagaagagattttgtgtataataataatgcaatgttcaatcctctcacaactcattacagctcagatatgaactgggcactcaacaatcatcaagaagaagaagaagagccacgaagaattgaaatttctgattccgagtcactagaaaacttgaaaagcagcgatttttatcaactgggtggtggtggtgctctgaattcgtcagaaaagccgagaaagatcgatttttggcgttcggggttgatgggttttgcgaagatgcagcagcagcaacagcttcagcattcagtggcggtgaagatgaacaataataataataacgatctaatgggtaataaaaaagggtcaactttcatacaagaacatcgagcattgttaccaaaagctttgattctgtggatcatcattgttgggtttataagcagtgggatttatcagtggatggatgatgctaataagattagaagggaagaggttttggtcagcatgtgtgatcaaagagctagaatgttgcaggatcaatttagtgttagtgttaatcatgttcatgctttggctattctcgtctccacttttcattaccacaagaacccttctgcaattgatcaggagacatttgcggagtacacggcaagaacagcatttgagagaccgttgctaagtggagtggcttatgctgaaaaagttgtgaattttgagagggagatgtttgagcggcagcacaattgggttataaagacaatggatagaggagagccttcaccggttagggatgagtatgctcctgttatattctctcaagatagtgtctcttaccttgagtcactcgatatgatgtcaggcgaggaggatcgtgagaatattttgcgagctagagaaaccggaaaagctgtcttgactagcccttttaggttgttggaaactcaccatctcggagttgtgttgacattccctgtctacaagtcttctcttcctgaaaatccgactgtcgaagagcgtattgcagccactgcagggtaccttggtggtgcgtttgatgtggagtctctagtcgagaatttacttggtcagcttgctggtaaccaagcaatagttgtgcatgtgtatgatatcaccaatgcatcagatccacttgtcatgtatggtaatcaagatgaagaagccgacagatctctctctcatgagagcaagctcgattttggagaccccttcaggaaacataagatgatatgcaggtaccaccaaaaggcaccaataccgttgaatgtgctcacaactgtgccattgttctttgcgattggtttcttggtgggttatatactgtatggtgcagctatgcacatagtaaaagtcgaagatgatttccatgaaatgcaagagcttaaagttcgagcagaagctgctgatgtcgctaaatcgcagtttcttgctaccgtgtctcacgagatcaggacaccaatgaatggcattctcggaatgcttgctatgctcctagatacagaactaagctcgacacagagagattacgctcaaaccgctcaagtatgtggtaaagctttgattgcattgataaatgaggttcttgatcgcgccaagattgaagctggaaagctggagttggaatcagtaccatttgatatccgttcaatattggatgatgtcctttctctattctctgaggagtcaaggaacaaaagcattgagctcgcggttttcgtttcagacaaagtaccagagatagtcaaaggagattcagggagatttagacagataatcataaaccttgttggaaattcggttaaattcacagagaaaggacatatctttgttaaagtccatcttgcggaacaatcaaaagatgaatctgaaccgaaaaatgcattgaatggtggagtgtctgaagaaatgatcgttgtttccaaacagtcaagttacaacacattgagcggttacgaagctgctgatggtcggaatagctgggattcattcaagcatttggtctctgaggagcagtcattatcggagtttgatatttctagcaatgttaggcttatggtttcaatcgaagacacgggtattggaatccctttagttgcgcaaggccgtgtgtttatgccgtttatgcaagcagatagctcgacttcaagaaactatggaggtactggtattggtttgagtataagcaagtgtcttgttgaacttatgcgtggtcagataaatttcataagccggcctcatattggaagcacgttctggttcacggctgttttagagaaatgcgataaatgcagtgcgattaaccatatgaagaaacctaatgtggaacacttgccttctacttttaaaggaatgaaagctatagttgttgatgctaagcctgttagagctgctgtgactagataccatatgaaaagactcggaatcaatgttgatgtcgtgacaagtctcaaaaccgctgttgttgcagctgctgcgtttgaaagaaacggttctcctctcccaacaaaaccgcaacttgatatgatcttagtagagaaagattcatggatttcaactgaagataatgactcagagattcgtttattgaattcaagaaccaacggaaacgttcatcacaagtctccgaaactagctctattcgcaacaaacatcacaaattcggagttcgacagagctaaatccgcaggatttgcagatacggtaataatgaaaccgttaagagcaagcatgattggggcgtgtctgcaacaagttctcgagctgagaaaaacaagacaacaacatccagaaggatcatcacccgcaactctcaagagcttgcttacagggaagaagattcttgtggttgatgataatatagttaacaggagagtagctgcaggagctctcaagaaatttggagcagaagtggtttgtgcagagagtggtcaagttgctttgggtttgcttcagattccacacactttcgatgcttgcttcatggatattcaaatgccacagatggacggatttgaagcaactcgtcagataagaatgatggagaaggaaactaaagagaagacaaatctcgaatggcatttaccgattctagcgatgactgcggatgtgatacacgcgacctacgaggaatgtctgaaaagtgggatggatggttacgtctccaaaccttttgaagaagagaatctctataaatccgttgccaaatcattcaaacctaatcctatctcaccttcgtcgtaa

SEQ ID NO: 4 - protein, 1176 aa, Arabidopsis thaliana
MSITCELLNLTSKKAKKSSSSDKKWLKKPLFFLILCGSLVIVLVMFLRLGRSQKEETDSCNGEEKVLYRHQNVTRSEIHDLVSLFSDSDQVTSFECHKESSPGMWTNYGITCSLSVRSDKQETRGLPWNLGLGHSISSTSCMCGNLEPILQQPENLEEENHEEGLEQGLSSYLRNAWWCLILGVLVCHKIYVSHSKARGERKEKVHLQEALAPKKQQQRAQTSSRGAGRWRKNILLLGILGGVSFSVWWFWDTNEEIIMKRRETLANMCDERARVLQDQFNVSLNHVHALSILVSTFHHGKIPSAIDQRTFEEYTERTNFERPLTSGVAYALKVPHSEREKFEKEHGWAIKKMETEDQTVVQDCVPENFDPAPIQDEYAPVIFAQETVSHIVSVDMMSGEEDRENILRARASGKGVLTSPFKLLKSNHLGVVLTFAVYDTSLPPDATEEQRVEATIGYLGASYDMPSLVEKLLHQLASKQTIAVDVYDTTNTSGLIKMYGSEIGDISEQHISSLDFGDPSRNHEMHCRFKHKLPIPWTAITPSILVLVITFLVGYILYEAINRIATVEEDCQKMRELKARAEAADIAKSQFLATVSHEIRTPMNGVLGMLKMLMDTDLDAKQMDYAQTAHGSGKDLTSLINEVLDQAKIESGRLELENVPFDMRFILDNVSSLLSGKANEKGIELAVYVSSQVPDVVVGDPSRFRQIITNLVGNSIKFTQERGHIFISVHLADEVKEPLTIEDAVLKQRLALGCSESGETVSGFPAVNAWGSWKNFKTCYSTESQNSDQIKLLVTVEDTGVGIPVDAQGRIFTPFMQADSSTSRTYGGTGIGLSISKRLVELMQGEMGFVSEPGIGSTFSFTGVFGKAETNTSITKLERFDLAIQEFTGLRALVIDNRNIRAEVTRYELRRLGISADIVSSLRMACTCCISKLENLAMILIDKDAWNKEEFSVLDELFTRSKVTFTRVPKIFLLATSATLTERSEMKSTGLIDEVVIKPLRMSVLICCLQETLVNGKKRQPNRQRRNLGHLLREKQILVVDDNLVNRRVAEGALKKYGAIVTCVESGKAALAMLKPPHNFDACFMDLQMPEMDGFEATRRVRELEREINKKIASGEVSAEMFCKFSSWHVPILAMTADVIQATHEECMKCGMDGYVSKPFEEEVLYTAVARFFEPC

SEQ ID NO: 5 - protein, 1036 aa, Arabidopsis thaliana
MSLFHVLGFGVKIGHLFWMLCCWFVSWFVDNGIEDKSGLLVGSVGDLEKTKMTTLKKKNKMWFWNKISSSGLKIPSFSYQFLGSVKFNKAWWRKLVVVWVVFWVLVSIWTFWYFSSQAMEKRKETLASMCDERARMLQDQFNVSMNHVQAMSILISTFHHGKIPSAIDQRTFSEYTDRTSFERPLTSGVAYAMRVLHSEREEFERQQGWTIRKMYSLEQNPVHKDDYDLEALEPSPVQEEYAPVIFAQDTVSHVVSLDMLSGKEDRENVLRARSSGKGVLTAPFPLIKTNRLGVILTFAVYKRDLPSNATPKERIEATNGYLGGVFDIESLVENLLQQLASKQTILVNVYDITNHSQPISMYGTNVSADGLERVSPLIFGDPLRKHEMRCRFKQKPPWPVLSMVTSFGILVIALLVAHIIHATVSRIHKVEEDCDKMKQLKKKAEAADVAKSQFLATVSHEIRTPMNGVLGMLHMLMDTELDVTQQDYVRTAQASGKALVSLINEVLDQAKIESGKLELEEVRFDLRGILDDVLSLFSSKSQQKGVELAVYISDRVPDMLIGDPGRFRQILTNLMGNSIKFTEKGHIFVTVHLVDELFESIDGETASSPESTLSGLPVADRQRSWENFKAFSSNGHRSFEPSPPDINLIVSVEDTGVGIPVEAQSRIFTPFMQVGPSISRTHGGTGIGLSISKCLVGLMKGEIGFSSTPKVGSTFTFTAVFSNGMQPAERKNDNNQPIFSEFRGMKAVVVDHRPARAKVSWYHFQRLGIRVEVVPRVEQALHYLKIGTTTVNMILIEQEIWNREADDFIKKLQKDPLFLSPKLILLANSVESSISEALCTGIDPPIVIVKPLRASMLAATLQRGLGIGIREPPQHKGPPALILRNLLLGRKILIVDDNNVNLRVAAGALKKYGADVVCAESGIKAISLLKPPHEFDACFMDIQMPEMDGFEATRRIRDMEEEMNKRIKNGEALIVENGNKTSWHLPVLAMTADVIQATHEECLKCGMDGYVSKPFEAEQLYREVSRFFNSPSDTES

SEQ ID NO: 6 - protein, 1080 aa, Arabidopsis thaliana
MRRDFVYNNNAMFNPLTTHYSSDMNWALNNHQEEEEEPRRIEISDSESLENLKSSDFYQLGGGGALNSSEKPRKIDFWRSGLMGFAKMQQQQQLQHSVAVKMNNNNNNDLMGNKKGSTFIQEHRALLPKALILWIIIVGFISSGIYQWMDDANKIRREEVLVSMCDQRARMLQDQFSVSVNHVHALAILVSTFHYHKNPSAIDQETFAEYTARTAFERPLLSGVAYAEKVVNFEREMFERQHNWVIKTMDRGEPSPVRDEYAPVIFSQDSVSYLESLDMMSGEEDRENILRARETGKAVLTSPFRLLETHHLGVVLTFPVYKSSLPENPTVEERIAATAGYLGGAFDVESLVENLLGQLAGNQAIVVHVYDITNASDPLVMYGNQDEEADRSLSHESKLDFGDPFRKHKMICRYHQKAPIPLNVLTTVPLFFAIGFLVGYILYGAAMHIVKVEDDFHEMQELKVRAEAADVAKSQFLATVSHEIRTPMNGILGMLAMLLDTELSSTQRDYAQTAQVCGKALIALINEVLDRAKIEAGKLELESVPFDIRSILDDVLSLFSEESRNKSIELAVFVSDKVPEIVKGDSGRFRQIIINLVGNSVKFTEKGHIFVKVHLAEQSKDESEPKNALNGGVSEEMIVVSKQSSYNTLSGYEAADGRNSWDSFKHLVSEEQSLSEFDISSNVRLMVSIEDTGIGIPLVAQGRVFMPFMQADSSTSRNYGGTGIGLSISKCLVELMRGQINFISRPHIGSTFWFTAVLEKCDKCSAINHMKKPNVEHLPSTFKGMKAIVVDAKPVRAAVTRYHMKRLGINVDVVTSLKTAVVAAAAFERNGSPLPTKPQLDMILVEKDSWISTEDNDSEIRLLNSRTNGNVHHKSPKLALFATNITNSEFDRAKSAGFADTVIMKPLRASMIGACLQQVLELRKTRQQHPEGSSPATLKSLLTGKKILVVDDNIVNRRVAAGALKKFGAEVVCAESGQVALGLLQIPHTFDACFMDIQMPQMDGFEATRQIRMMEKETKEKTNLEWHLPILAMTADVIHATYEECLKSGMDGYVSKPFEEENLYKSVAKSFKPNPISPSS

SEQ ID NO: 7 - DNA, 4595 bp, Arabidopsis thaliana
  source 1..4595: mol_type = unassigned DNA
atgtctataacttgtgagctcttgaatcttacttcaaagaaagctaagaagtcgtcgagcagtgacaagaaatggctaaagaagcctctcttcttcctgattttgtgtggctctttggtaattgttttggttatgttcttacggttaggtagaagtcagaaggaggagacagattcttgtaatggagaagagaaagtgttgtatagacatcaaaatgtcacaagaagtgagattcatgatttggtctctttgttctctgattcagatcaggtaattgcatgaattgacttgtatttgttgaaattgagcttttgaatacgcaccagatttgccttaaagtgtaacagtttctctgtatttgttgtaggtaacatcctttgaatgtcataaggaatcaagccctggaatgtggacaaactatggtattacatgttccctgagtgtgcgttctgataaacaagagactagagggcttccctggaatcttggcttaggacattctatctcatcaacatcttgtatgtgtggtaatcttgaaccggtaagataatattcgattcgacaacatgtgaaggaaatgctttaagatttggtgtttagctagttctcacaaaagtttattgtgaatgtgtttggttatgagtagattttacagcaacctgaaaaccttgaggaagaaaaccatgaagaagggctggagcagggtttgtcatcgtatttaagaaatgcatggtggtgtctaatccttggtgtgttagtgtgccataagatttatgtatctcattctaaagcacgaggtgagaggaaagagaaagtacatctgcaagaggctttagctccaaagaagcagcaacaacgtgctcagacttcttctagaggggctggaagatggaggaagaatatccttctccttggtattttaggaggagtttccttctctgtttggtggttttgggacactaatgaggagatcataatgaaaaggagggagactttggcaaacatgtgtgacgaacgagcacgtgttttacaagatcagttcaatgttagcttgaaccatgttcatgccttgtctattcttgtatctacatttcatcatggtaaaatcccatctgccattgatcaggtgatgtttttttcttactgctaaatacattttgtgtctcaagtttatgtttaaatcatcaacttctgttacatttacagagaacatttgaagaatatactgagagaacaaactttgagaggccacttactagtggtgtagcgtatgctttgaaagtcccacactcagaaagagagaaatttgaaaaggagcatggatgggcaataaagaaaatggaaactgaggaccagacagttgtacaagattgtgttcctgaaaactttgatcccgcaccgattcaagacgaatacgcgccagttatatttgctcaagaaactgtttcccatattgtatcggtcgacatgatgtctggagaagtcagtaacgtctaaaagtttcttgaactattttgccaaaccaatgtccttaaaagagaattcaaaagtctaactattttgcaggaagaccgtgaaaacatcttacgggcaagggcatcaggaaaaggagtgttaacatctccatttaagcttcttaagtcaaatcatcttggtgttgtgttgacctttgctgtctatgacacgagcctaccgcctgatgctacagaagaacagcgtgttgaagcaactattgggtactacttctacttaaaatgattctaggactgaagaaattgaacctatgtaacaaagaatgatctctggaccagaaaatattaataagatacacttaaaaacaggtaccttggtgcatcatatgatatgccatcgctggtggagaaacttcttcaccaacttgccagcaaacagacaattgctgtggatgtttacgacacaactaacacttcaggtctaataaaaatgtatggctcagaaattggggatataagtgagcagcatataagtagccttgattttggtgatccatcaaggaaccatgagatgcattgcaggttagttagctctaactgttatggtacatttttataagatatgtttcacttctctgttttctgaagtatgaaagtggcatttttcatttacaggtttaagcataaacttcccattccctggacagcgataacaccgtcgatcttagttctggttattacttttcttgttggttatattttatatgaagccatcaaccgaattgcgacagttgaagaggattgtcagaagatgagggaactcaaagctcgtgctgaggccgctgacattgcaaagtcacaggtgatctttgtgaatcatatagtctcaaaagctttacttgttttttcactgaaatgcttcttattttgcagttcctagcaactgtttctcatgagatacggactccgatgaatggagttttaggtactttttctacttatccttggtcaatattgcatgttctcttaaaatcagctgaaacgtttaagcattttgttacaggaatgctgaaaatgctgatggacaccgatcttgatgcgaagcagatggactatgcgcaaactgctcatggcagtgggaaggatcttacatcactaataaatgaggttcttgatcaggcaaagattgaatccggaaggctcgagcttgaaaatgtgccttttgatatgcgttttattcttgataatgtttcatctctcctctctggcaaggcaaatgaaaaaggaattgaggtataattataaactgcatgaccttctactttcttaatgttttcatatggcaaacaaattccatatgtaatgaaatgttgacttcttgttacagttggccgtttatgtttctagtcaagttcctgatgttgtagtcggtgatccgagtcggttccggcagatcattacaaacctggttggaaactcaatcaaagtaatttactccttacttcttacagaacaacaggcttcacgaatctctactattacagtactcatttgttatttacttaataacagttcacacaggaaaggggacacatatttatctcagtgcaccttgcagatgaggtaaaggagcctcttactattgaagacgcagtgctaaaacagcgactagctttaggatgcagcgagtccggtgagacagttagcgggtttcctgcggtaaatgcatggggaagctggaagaatttcaagacatgttacagtactgagagtcagaattctgatcaaatcaaattgctagttacagtggaggacactggagttggcatacctgtggatgcacaaggccgaatcttcacaccttttatgcaagccgacagttccacatcgcggacttatggtggaactggcataggtttgagtataagcaaacgtttggttgaactcatgcaaggagagatggggtttgtgagtgagcccgggataggcagtactttttcatttactggagttttcgggaaagcagaaacaaatacgtcgattactaagctggaacgattcgatctagctattcaggagtttacaggattgagagcattagttattgataacagaaacattagagcagaggtcaccaggtacgaacttcggagactgggaatatctgcagacattgtttcaagtctgagaatggcatgcacttgttgtatcaggtactttacgtacattagtgtctgtctgtctttagagattatagtgagttcactaaaagcgttttattgttctggaatctttgcagcaaattagaaaatttggctatgattctaatagacaaagacgcctggaacaaggaagaattttcagtacttgacgagttgtttacccgaagcaaagtaacctttacaagagtcccaaagatttttcttttggcaacttctgcaactcttactgagcgcagtgagatgaagtctactggtctcatcgatgaggtggtgataaagcctcttcggatgagtgtcttaatatgttgcttgcaagaaacccttgtcaatggcaagaagaggcaaccgaacagacagcgaagaaatcttggacacttgctaagagaaaaacagattctggttgtggatgataatcttgtgaacagacgagttgcagaaggtgcacttaagaaatatggagctattgttacatgcgttgagagtggcaaagctgcattggcaatgcttaagccgcctcataacttcgatgcttgcttcatggatctccagatgcctgaaatggatgggtagataactatttttcaatcttatctcttcagcttgttcatttcttggatgtcgtccttggtaactttataattttttgcgcagatttgaagcgacaaggagagtccgtgagctggagagggaaatcaataagaaaatagcttctggagaagtttcagctgaaatgttctgtaaatttagtagttggcacgtcccgatattagcaatgacagcagatgttattcaggctactcatgaagaatgcatgaaatgtggaatggatggttatgtatcaaaaccgtttgaagaggaagtgctctacacagcggtagcaagattctttgaaccttgttaa

SEQ ID NO: 8 - DNA, 4248 bp, Arabidopsis thaliana
  source 1..4248: mol_type = unassigned DNA
atgagtctgttccatgtgctagggtttggtgtcaagattgggcatctcttctggatgctatgctgctggtttgtttcttggttcgttgataatgggatcgaggacaagtctggtcttttagttggctctgtcggtgatcttgagaagactaagatgactacgttgaagaagaagaacaagatgtggttctggaataagatctctagcagcggactcaagatcccgagtttctcttatcagtttcttggctctgttaaattcaacaaggcgtggtggaggaagcttgtggtggtttgggttgtcttctgggtcttggtctctatttggacgttttggtactttagctcgcaagctatggagaagaggaaagagacgctagctagtatgtgtgatgagagagctcgtatgctgcaggatcagttcaacgttagcatgaatcatgttcaagccatgtctatcttgatctcaaccttccaccatggcaagattccttctgctatcgatcaggtattctcgattccgcatcttcttgtcttctaatctgtttggattgatgcctgagatctagtcttgcttgtagtacataagttgttcctacggtgtagtatatgaacggtccttggatgatggtagcttttttcattcaacttgctctcgaatttcagttaggatttacaattttgctgattgttgcttacagtctttgtatttagctaacacttggtagcttgattcctatcttcttgagtattagtgaacagtaactaacattaagaagctttgtgtagagaacattctcagagtacactgatagaacttcctttgagaggcctcttactagcggggtagcttatgctatgagggtgctccattcagagagggaagagttcgagaggcaacaaggttggactattaggaagatgtattctcttgaacaaaacccagttcacaaggatgactatgacctggaagctttggaaccatcccctgtccaagaagagtacgctccagtcatctttgctcaggacactgtttctcacgttgtttctctcgatatgctgtctgggaaagtaagcttctttactggttctaattttactgtttttatgtaatttactatcatggtctacactcactaccgggcgaatcctgccgacatggctagttcatatttcaatctcggcttaccgattaagttgttttgatcattttatttaaagtttagagcactgttttaactgatacttattctcactcaattcctcttgataggaagatcgtgaaaacgttttgcgggccaggagttcaggtaaaggggttttgacagctcctttcccattgataaagacaaatagacttggggtgatcctgacatttgcagtgtacaagagagatctcccctccaatgcaacgccaaaagagagaattgaggctactaacgggtgtgtagcataggcgtctaaataaataataacgcataagttttacaacattatttttgctcattcatctcttttgatgcccctgaaggtatctcgggggagtgtttgacattgagtccctggtagaaaacttgcttcaacagctggctagcaagcaaacgattcttgtcaatgtgtacgatatcaccaatcactctcaaccgattagcatgtatggtacaaatgtgtcggctgatgggttggaacgtgttagtccactaatctttggcgatccattgagaaagcatgagatgcgttgcaggtacttgcagttggcacatacatatgtctgtaatttcttcttgtttgcaagaatccaggtgctaacattttgttgtgagcttcttcctctttgtagatttaagcagaaaccaccatggccagtgctatcaatggtgacatcattcggtatccttgtgattgcgttacttgttgcacatataatccacgcaaccgttagtcgaatacacaaagttgaagaagattgtgataaaatgaagcagctcaagaaaaaggctgaagcagcagatgttgcaaagtcacaggtaaatacatcatcctcagttgataaatttccacagcatattaaactttctatgacagagagaagagttgtaatctaacttttttttatgcagttccttgccactgtttcacatgaaatcagaactccaatgaatggtgttctaggtgagtatcgaaatggccactatgttgcctccttctctcacctatgcccatgaatattttctgaagaacaaacttaaccaaatattctcatgttgactttgatctggggtgtaggaatgttgcatatgcttatggacacagagttagatgttacgcaacaggattatgttaggaccgcacaggcaagtggaaaagctttagtctcgctaataaatgaggttttggaccaagcaaagattgaatctggaaagcttgaacttgaggaggtgcggtttgatttgagaggaatattagatgatgtcctgtcactcttctctagcaagtcccaacaaaagggggtggaggtaacttactatatgatctgcaaagcaagggttgtaactgatagcaagtctttcttactgattttttgagtgttgctgatgaacgcagttggcagtatacatatctgatcgtgttccagatatgttaattggtgatcctgggaggtttcgacaaatactcacaaatcttatgggtaattccattaaggtaaacttttttattatgtttttttctgaccattccttgcatccgagttgatcaacggagctgatcatttatatatattctggcagttcactgagaaaggacacatctttgtaactgttcatttggtggatgagctatttgaatctatcgatggagagacagcatcatctccggaaagtacactgagtgggcttccagttgcagaccggcagaggagctgggaaaactttaaagctttcagctccaacgggcatcggagctttgaaccatctccccctgatataaacctaatcgtctcagttgaggatactggcgtagggatccctgtagaagcgcagtcccgtatttttacgcctttcatgcaagtcggaccatccatatccaggacgcatggaggcacaggaattggacttagcataagcaaatgtctagttggactgatgaagggagaaattggattctcgagtactcccaaggttgggtccacattcacatttactgctgtattttccaatgggatgcaaccagctgaaagaaagaatgacaacaaccagcccatattctcggaattccggggcatgaaagctgtggttgtggaccataggcctgcaagggcaaaagtctcgtggtaccattttcagcgtcttggaattcgagtcgaagtagttccacgtgttgaacaggctctacattatctgaagattggtactaccactgtgaatatgatactcatagagcaagaaatatggaatagggaagcagatgatttcattaaaaagctacagaaagaccctcttttcctttctcctaagttgattttgttagcaaactcagtagaatcgtcaatatcagaggctttatgcaccggtatagatcctccaatagtgatagtgaaaccattgagggcgagtatgctagcagcaactttgcagaggggattgggtattggaatcagagaaccacctcaacacaagggacctcctgctttgattctcaggaatcttctccttggtagaaaaattttaatcgtggatgataacaacgtaaacctcagagtggcagcgggagctctgaaaaagtacggagctgatgtggtctgcgctgagagtgggataaaggcaatctcattgcttaagccacctcacgagtttgatgcttgcttcatggacattcagatgccagaaatggatgggtatgcctgattggtatactagtttttttgaaaagttcgaaatatgtaataagaaaattgaaatgtttttctgccctgtctttctgcagatttgaagctacaaggagaatacgagatatggaagaggagatgaacaagagaataaagaatggggaggctttgatagtagagaacggtaacaaaacaagctggcatcttccggtattagcaatgacggcagatgtgatccaagcaacgcatgaggaatgtctgaagtgtggaatggatgggtatgtatcaaaaccatttgaagcagagcagctgtacagggaagtttctcgctttttcaattcgccttcagatacagaatcataa

SEQ ID NO: 9 - DNA, 4685 bp, Arabidopsis thaliana
  source 1..4685: mol_type = unassigned DNA
atgagaagagattttgtgtataataataatgcaatgttcaatcctctcacaactcattacaggtaactaaaataatttctccatgtgcttgcttattagtcgttcttcctaatgttatgtttctctctgtgttctttctttctttggtcaaagctttaattttttttctattgttggatttgagacagtgaacatagctatgttcttgttccaataataaacaatcacgcctgtaaagagcttatgattgattagtgtgttttttagtattaattaatttctctgacaataattacttagtttttaattcttctctgtaagaaacctttggaaactgagcaaagttgcttcttttgagaaccatgcgtttctttctctcttttgttcttgaattcgcaaaaacatgtcctttttcgtctacaggtttctagggtttgtttctgtactataaactatgtttatggtaacattcttaatcataactacactaccaatgcttttatgttatatgtatgcaaaaaaggctctaacttttgttttctttcactattgtttcttcttttgttctctattgttgtagctcagatatgaactgggcactcaacaatcatcaagaagaagaagaagagccacgaagaattgaaatttctgattccgagtcactagaaaacttgaaaagcagcgatttttatcaactgggtggtggtggtgctctgaattcgtcagaaaagccgagaaagatcgatttttggcgttcggggttgatgggttttgcgaagatgcagcagcagcaacagcttcagcattcagtggcggtgaagatgaacaataataataataacgatctaatgggtaataaaaaagggtcaactttcatacaagaacatcgagcattgttaccaaaagctttgattctgtggatcatcattgttgggtttataagcagtgggatttatcagtggatggatgatgctaataagattagaagggaagaggttttggtcagcatgtgtgatcaaagagctagaatgttgcaggatcaatttagtgttagtgttaatcatgttcatgctttggctattctcgtctccacttttcattaccacaagaacccttctgcaattgatcaggtcagtgtgtgctttattaaaagctcaaagctttggtgaaatttttgattcataaatgcaaagttgactttttttggaaccagaagtttttgaaatttactcttttatgtttggttattgagtaaatgaagggaaatttaggcctttgctttactcattggtttgtggctaattttgcaggagacatttgcggagtacacggcaagaacagcatttgagagaccgttgctaagtggagtggcttatgctgaaaaagttgtgaattttgagagggagatgtttgagcggcagcacaattgggttataaagacaatggatagaggagagccttcaccggttagggatgagtatgctcctgttatattctctcaagatagtgtctcttaccttgagtcactcgatatgatgtcaggcgaggtaatataaacattatcagacacttatggttttgttttttcggttaagtatccgacctgcacctcttctgaattttggctatcttttctgcaggaggatcgtgagaatattttgcgagctagagaaaccggaaaagctgtcttgactagcccttttaggttgttggaaactcaccatctcggagttgtgttgacattccctgtctacaagtcttctcttcctgaaaatccgactgtcgaagagcgtattgcagccactgcagggtaaagttcccatctgattactggagcaaacttttgcttgtgatcatgtaaaaagagattttcgagatttcatgattggtttatgtgtgtgatcatgtaaaggaaagaggttttttgagatttcaagattggtttgtgtgtaaagtaaagattgatgcttgaatttgaaatgcaggtaccttggtggtgcgtttgatgtggagtctctagtcgagaatttacttggtcagcttgctggtaaccaagcaatagttgtgcatgtgtatgatatcaccaatgcatcagatccacttgtcatgtatggtaatcaagatgaagaagccgacagatctctctctcatgagagcaagctcgattttggagaccccttcaggaaacataagatgatatgcaggtaccacacagaaaatcttaaagctaaaatactctctgatatctaaaaactgatatcttgtttgaacaatttcgagtgcaggtaccaccaaaaggcaccaataccgttgaatgtgctcacaactgtgccattgttctttgcgattggtttcttggtgggttatatactgtatggtgcagctatgcacatagtaaaagtcgaagatgatttccatgaaatgcaagagcttaaagttcgagcagaagctgctgatgtcgctaaatcgcagtttcttgctaccgtgtctcacgagatcaggacaccaatgaatggcattctcggtactacatcttcaaacaaattccagtactgttgcctttcatctttggtttctcaaactgcctcaaagattctgttttaaaaattttctgcaggaatgcttgctatgctcctagatacagaactaagctcgacacagagagattacgctcaaaccgctcaagtatgtggtaaagctttgattgcattgataaatgaggttcttgatcgcgccaagattgaagctggaaagctggagttggaatcagtaccatttgatatccgttcaatattggatgatgtcctttctctattctctgaggagtcaaggaacaaaagcattgaggtaagttttcaatccaaggaataaagatacatctatttggagttgtggctgctaatttcttgatttcaaatttggcagctcgcggttttcgtttcagacaaagtaccagagatagtcaaaggagattcagggagatttagacagataatcataaaccttgttggaaattcggttaaagttagtccctatttcaatctctgcctttgttggcagcttgtgtatatgatttttgtttgatcaagatgttgtctttttgacagttcacagagaaaggacatatctttgttaaagtccatcttgcggaacaatcaaaagatgaatctgaaccgaaaaatgcattgaatggtggagtgtctgaagaaatgatcgttgtttccaaacagtcaagttacaacacattgagcggttacgaagctgctgatggtcggaatagctgggattcattcaagcatttggtctctgaggagcagtcattatcggagtttgatatttctagcaatgttaggcttatggtttcaatcgaagacacgggtattggaatccctttagttgcgcaaggccgtgtgtttatgccgtttatgcaagcagatagctcgacttcaagaaactatggaggtactggtattggtttgagtataagcaagtgtcttgttgaacttatgcgtggtcagataaatttcataagccggcctcatattggaagcacgttctggttcacggctgttttagagaaatgcgataaatgcagtgcgattaaccatatgaagaaacctaatgtggaacacttgccttctacttttaaaggaatgaaagctatagttgttgatgctaagcctgttagagctgctgtgactagataccatatgaaaagactcggaatcaatgttgatgtcgtgacaagtctcaaaaccgctgttgttgcagctgctgcgtttgaaagaaacggttctcctctcccaacaaaaccgcaacttgatatgatcttagtagagaaagattcatggatttcaactgaagataatgactcagagattcgtttattgaattcaagaaccaacggaaacgttcatcacaagtctccgaaactagctctattcgcaacaaacatcacaaattcggagttcgacagagctaaatccgcaggatttgcagatacggtaataatgaaaccgttaagagcaagcatgattggggcgtgtctgcaacaagttctcgagctgagaaaaacaagacaacaacatccagaaggatcatcacccgcaactctcaagagcttgcttacagggaagaagattcttgtggttgatgataatatagttaacaggagagtagctgcaggagctctcaagaaatttggagcagaagtggtttgtgcagagagtggtcaagttgctttgggtttgcttcagattccacacactttcgatgcttgcttcatggatattcaaatgccacagatggacgggtaactaaatcacaactaatactatttttggattattcggtttttgaattgttcggtatgaaaaatttcccgccaaattataactgacttggtttggttcggattcggttaaaatcttttataaacttcattgattctcagatttgaagcaactcgtcagataagaatgatggagaaggaaactaaagagaagacaaatctcgaatggcatttaccgattctagcgatgactgcggatgtgatacacgcgacctacgaggaatgtctgaaaagtgggatggatggttacgtctccaaaccttttgaagaagagaatctctataaatccgttgccaaatcattcaaacctaatcctatctcaccttcgtcgtaa

SEQ ID NO: 10 - DNA, 2000 bp, Arabidopsis thaliana
  source 1..2000: mol_type = unassigned DNA
taatttgaatatttatattcaatttcatacatattattttaaaatttataattttatatactaaaataattaataacatattagattttttaatgtgtatgatgtgattcaatttttaatacaagttggaaaatcctagaattgacgggtttaaatcaatattaatacaagatggtatagtacaggtgaaactctaaaattaacaatcaaaatacaagtatataattttaaacattaaacaatataaataatatcaacaaaacatatacaactagcagtttactatacatttaaaatcaaataaaataatatcatatatttgtaatcaactttacaataaaagtttttagtttaactaaaatataagcctacctgctgtataatgcaagttaaaattataaataagcagattaccatcacgatggatgtaccttctcacatacacacctatcaaaaacttaaaacctttcaaaacctcttggtatattcaaattaggattcttcattcatagaatcattattccaagggttttgggatgtcttatgaaatgattttggcagaagaggatgtaaatttttttgaataaaaggtattcaacaaaacatatgttttattgggattttttagataattaaatcagagaatagggctgggcatatgagtaacccattcgagttcgggtagaacccgttcgggtttgggattaatgggtatatgatttactacctaatagaataattctaaatattcggttcgggtcgggtccagttgggtttgggtcagtttggataaaaagtttcaggccctaaaaatactcaaaaaaatagaaacgagtattttgtatgtagatagtgcgtctctaaacaaaaaaaacttttaaatttattaagttttaataaatttaattatatttgaatatattttactaaattttgacaaatataacaaataattttgataaacataacaaacataacaaagtatacaatgtaatttatacaaaatattaatgcccgtgctatacagcacgggttatgatctagtcctaatatattttcaaagtcaaatacaattttaaacagaacttcttacataaaaataatcagaatcataaatgtaactgggagacaaagatacataaactgataaaaagaaataagatgtacatatagaatcaaatcaatacccacttctcgtaaaacccactcatcgttatattttatacaagataagataagacttaagagtagatgttaaactatgctaatgaagcaatttaaaaacaccaaaaagaccaaacaaaaaacaaataacaatgagtaggttttaggagaagcatgtattgattcttggtatagttgagtttatttatattttcattagccaatcactttttctaatttaactaaaaatgttataataccttccccacctagatgatgagataatcatggacccaaaaacctcagtttttaaaaatgtttattcaccaaaagacaaaaacaataataataatcaaaagtagagtaaaagaagagagaagtgacattctttttctttagctcataatttctctaacaaatgcttcttttatccaaactctgatcattgagtccagttgggtaatgagagacaccagctgggtttcttttgtttaagctcacaaacatctaaaacagagaaagctttgtcatttttctttaccctgaaaccatgaaagaagataaagctttgtcctttttctcttttccgtttctttactctcaacaatgtcttctttctcccattgcaacaaccactgggtttggttcatcttgtcctcttgcgtcttaagtaagaaaaaagaaaaaagctttgcgtttgggactatgtcacagcttcttgttcttcttgtggatggtttcaagaactcaactatgagattgttgttgatagagttttagttttgggtttgaagagatctgaatttcttcagctgaatctgagattaggagtcgaa

SEQ ID NO: 11 - DNA, 2000 bp, Arabidopsis thaliana
  source 1..2000: mol_type = unassigned DNA
tctggatccatctctttcttcgtcttcctttttgatccttagctcttgcgtcgtgctttttcaacaggcgagagaggatcgagagggttagataatttactgaaaaattatacgaatggaccaagactagagatactgaaatgagtgtttgacgatactttaaatgggccttatatttttcaagttgggcccgttaatatatgggacaatcaacagtatagctcaattggtaaattgactaacttcatttgattcctacaccatttaaagatttttaatcaaattctgatataactaagataatattttaaagcatttaactaacatggaaaattatgaaatgaaatattaattttggatagaaaatttaaaacatttatctttgtgaaataaaaagaaagtctcttataatataacttgtaaagtaatcctttaaattctaaaagatctaaaacaaaatgagttaaatcctatgtctatgtgttgaatctggaagatcgaggtggaagaaaaaggatcatgattcatgggtgagagaacaaccgttgattggtgtgtttcaactttcaagcaatcccattttaaaaaaataaatttatgtaagcaattgcaaacaaaatgaaatgattatgctttaagatatcgacacgtgtaaaacagatgacgtaccaagaaaataaatgattctgtattttgttttagccatttgttttttctttaaaggaaagtgtatgttgaaattgaaaaaacaaaccgagttatatatatagagttatttgactaaagaaggaagctttggttaaactaacttgtataattcttttgtgtagtttagttttgataaaatggccaaacaattttggtcactttaccaactactgcaaaaaaaccattttcataataataaattgtaacacgccttttatttttattattggagggtgagaactgagaccatatatgcctttgttcccttcttgtgggtccctttagcctttttttttcttttgcctgcgccgtttagtgttttttcttttatatataaatagaggtaagttattttaccaatcatttggtgttattaatagtagtattaatgaggttctaaacttgtttataaaaatcagatcttactaaacaaactcaaagaatctcaactatgaacatttttcttctagaatcgagttttaacatcatcatcagcaggatataattttttgtgtttgaattttatctgtattttttttaggagtacataagcagcttaagattaaaatatatattaatggaagtatgaatgaaaaaagaaaagaaagataatcagataatagagagagagagagagagaaggaagcagacatttgtcttccaccatattcttctcatcttttccgatgatagattcttcttcttcttcttcttctactactacttctgctcctctttcttaagtttctccatttgataattcactcaagtctccagctcctcttcttcttctcgttgctttattcgctctctttcactcatcatcatcacactcaaagatttctgttgtattggagcaagctctagcgaattttcgtactccccttgtagaattttagtttcctgttttagatttcgaggaatctcgctttggaaactttaagctcctgttcaaagtttctacctttgtttattttgtttttgttttcttctctcttatctttcgtcatctttgattaagtaaccactttgatctctcaatttcagcctttgggttgtctaatctgacacgttcctctgtcttagctttccttttctcttctcgtttgtgggaagcctacaaggattaattattaaatacccaaccccaaaaaattcttcctttttgattctctgattcgtccctttttgtacatggtttctgtttggttgttagagcttcttgctgagttgaattgagctggttgcaatcactctgtttcttgggggttgatcgtgtattcaagtggtgg

SEQ ID NO: 12 - DNA, 2000 bp, Arabidopsis thaliana
  source 1..2000: mol_type = unassigned DNA
ccaattcacgttaaatctatctcttgctcttgcttcctccaaaaaaaaaaaaaaatcattcccagatccatcgatatgaaattgtatagaaaaaatggtattcgatccaagtttattgtcttctatttttcttaggttaatttcactttattccagattcattgtttgttttttctttctcggaagagcacaatgtgagtttcactggcctctgttataaacatatatagaaatctgtaacaaaaatcattactaaaattctgtgacatgtgcagcgatcaaagaatcaatagcggaaaaagaaactacactgcattcatctatgactgaaagcttctgatcaagccatgaaattaaggtatcccaaacacgtatcttctctatgtttatcaatcttgctttaagttctaattctgcatatttcaaaggaaccatacaagtgttcctaaaatccatttgaatattcaaaaacttctctcaaatatcatgtagttatagaagctactgtctctaagcgcacgagagaaagctacacaacccacgtcagtttccatctacacatataaggtaataataatattttcatgtatctttaataatagctctatgtttttttctgtatttttcattataaaactcataactatgttatcatttaatatggtactaatttaatgggattgatttactattgcctcaaacatgtaataatttaatgattttttgtttttaacgtttttagaaattcatgagcattttaaatttgtggttaggtcataacaatttgctattacaaaaaaaagaaacactctaaataatataaaaaatagtttaccgtataatactagtagtaaataaataatttgattgttattcataaattttgaattctaaaatctcctgaatcaactcatgcaattgtcttaagaattacacgtggataaatcatgggcttatgagtcaggcccatttaaccggggtattttcgtagttaagagactagaatggtgggtatttcaggtaaaaggtctatggggccagatctgcgctttgtcgcgatgtcattatcgccaaagatatgcgatagcgactctcgtacaaagtctctcactcacctatattttttgttttcttatatttcaacaaaaaaacgttttattttccttttggtgtaagtaaaaaaacaaaacaaaacgttttatttctaaagttcagaaaacttatttataccaaggaaaaaatagataataaattttgagaagttggtgactatatattacttcacttattcaagaaatttaaacatggtaaatgttactttaaatgttaaatgatgtataagaaatgtaatgaaattgaataaatgtagttttaaagatgttttaattagtaagacaaacctagttagtgtcacaataattatatttttttttttgtcatccaaaattattaaagctcaagtaaaccaatcctgagggatattatttacaaatgtgatatgatgcggttcggtgcggatcttccgcgccaaattatacgcttttatattagcattataaaaaattatagataaagagaagtttgtgaattcttcattgtcgctttgcaatttctctaaatacacagtaaataccgacaattcggttagagaaaatatatctatttcgtataataatgttaactttgaggagattttgggtaaaataataacttttgttggatggatcatatcatgagccattaagaaaaagtccaaaacttttcttcttcaaagttggactcaagttagaaaaagaaaaaagagctagagagatataaaaatgaaaagaaagttcatggcaaaaaactgatatagacagagacacagagagagagaaacgtatctgaagaaaatctaaaaaattcgattcaatttttttcttacttttaaaagcaaaaaatctcactaaaacaaaagaagaagaaagaagaaagaaaatggaatacctacatttgaagtg

SEQ ID NO: 13 - DNA, 2000 bp, synthetic construct
  misc_feature 1..2000: note = Modified AtCHK2 promoter with SNP
  source 1..2000: mol_type = other DNA
taatttgaatatttatattcaatttcatacatattattttaaaatttataattttatatactaaaataattaataacatattagattttttaatgtgtatgatgtgattcaatttttaatacaagttggaaaatcctagaattgacgggtttaaatcaatattaatacaagatggtatagtacaggtgaaactctaaaattaacaatcaaaatacaagtatataattttaaacattaaacaatataaataatatcaacaaaacatatacaactagcagtttactatacatttaaaatcaaataaaataatatcatatatttgtaatcaactttacaataaaagtttttagtttaactaaaatataagcctacctgctgtataatgcaagttaaaattataaataagcagattaccatcacgatggatgtaccttctcacatacacacctatcaaaaacttaaaacctttcaaaacctcttggtatattcaaattaggattcttcattcatagaatcattattccaagggttttgggatgtcttatgaaatgattttggcagaagaggatgtaaatttttttgaataaaaggtattcaacaaaacatatgttttattgggattttttagataattaaatcagagaatagggctgggcatatgagtaacccattcgagttcgggtagaacccgttcgggtttgggattaatgggtatatgatttactacctaatagaataattctaaatattcggttcgggtcgggtccagttgggtttgggtcagtttggataaaaagtttcaggccctaaaaatactcaaaaaaatagaaacgagtattttgtatgtagatagtgcgtctctaaacaaaaaaaacttttaaatttattaagttttaataaatttaattatatttgaatatattttactaaattttgacaaatataacaaataattttgataaacataacaaacataacaaagtatacaatgtaatttatacaaaatattaatgcccgtgctatacagcacgggttatgatctagtcctaatatattttcaaagtcaaatacaattttaaacagaacttcttacataaaaataatcagaatcataaatgtaactgggagacaaagatacataaactgataaaaagaaataagatgtacatatagaatcaaatcaatacccacttctcgtaaaacccactcatcgttatattttatacaagataagataagacttaagagtagatgttaaactatgctaatgaagcaatttaaaaacaccaaaaaggccaaacaaaaaacaaataacaatgagtaggttttaggagaagcatgtattgattcttggtatagttgagtttatttatattttcattagccaatcactttttctaatttaactaaaaatgttataataccttccccacctagatgatgagataatcatggacccaaaaacctcagtttttaaaaatgtttattcaccaaaagacaaaaacaataataataatcaaaagtagagtaaaagaagagagaagtgacattctttttctttagctcataatttctctaacaaatgcttcttttatccaaactctgatcattgagtccagttgggtaatgagagacaccagctgggtttcttttgtttaagctcacaaacatctaaaacagagaaagctttgtcatttttctttaccctgaaaccatgaaagaagataaagctttgtcctttttctcttttccgtttctttactctcaacaatgtcttctttctcccattgcaacaaccactgggtttggttcatcttgtcctcttgcgtcttaagtaagaaaaaagaaaaaagctttgcgtttgggactatgtcacagcttcttgttcttcttgtggatggtttcaagaactcaactatgagattgttgttgatagagttttagttttgggtttgaagagatctgaatttcttcagctgaatctgagattaggagtcgaa

SEQ ID NO: 14 - DNA, 34 bp, synthetic construct
  misc_feature 1..34: note = pAtCHK2_F
  source 1..34: mol_type = other DNA
taatttgaatatttatattcaatttcatacatat

SEQ ID NO: 15 - DNA, 23 bp, synthetic construct
  misc_feature 1..23: note = pAtCHK2_R
  source 1..23: mol_type = other DNA
ttcgactcctaatctcagattca

SEQ ID NO: 16 - DNA, 26 bp, synthetic construct
  misc_feature 1..26: note = pAtCHK4_F
  source 1..26: mol_type = other DNA
ccaattcacgttaaatctatctcttg

SEQ ID NO: 17 - DNA, 26 bp, synthetic construct
  misc_feature 1..26: note = pAtCHK4_R
  source 1..26: mol_type = other DNA
cacttcaaatgtaggtattccatttt

SEQ ID NO: 18 - DNA, 26 bp, synthetic construct
  misc_feature 1..26: note = AtCHK2_F_CDS
  source 1..26: mol_type = other DNA
atgtctataacttgtgagctcttgaa

SEQ ID NO: 19 - DNA, 24 bp, synthetic construct
  misc_feature 1..24: note = AtCHK2_R_CDS
  source 1..24: mol_type = other DNA
ttaacaaggttcaaagaatcttgc

SEQ ID NO: 20 - DNA, 55 bp, synthetic construct
  misc_feature 1..55: note = AtCHK2_F_CDS_attB1F
  source 1..55: mol_type = other DNA
ggggacaagtttgtacaaaaaagcaggctatgtctataacttgtgagctcttgaa

SEQ ID NO: 21 - DNA, 53 bp, synthetic construct
  misc_feature 1..53: note = AtCHK2_R_CDS_attB2R
  source 1..53: mol_type = other DNA
ggggaccactttgtacaagaaagctgggtttaacaaggttcaaagaatcttgc

SEQ ID NO: 22 - DNA, 32 bp, synthetic construct
  misc_feature 1..32: note = AtCHK4_F_CDS
  source 1..32: mol_type = other DNA
atgagaagagattttgtgtataataataatgc

SEQ ID NO: 23 - DNA, 22 bp, synthetic construct
  misc_feature 1..22: note = AtCHK4_R_CDS
  source 1..22: mol_type = other DNA
ttacgacgaaggtgagatagga

SEQ ID NO: 24 - DNA, 61 bp, synthetic construct
  misc_feature 1..61: note = AtCHK4_F_CDS_attB1F
  source 1..61: mol_type = other DNA
ggggacaagtttgtacaaaaaagcaggctatgagaagagattttgtgtataataataatgc

SEQ ID NO: 25 - DNA, 51 bp, synthetic construct
  misc_feature 1..51: note = AtCHK4_R_CDS_attB2R
  source 1..51: mol_type = other DNA
ggggaccactttgtacaagaaagctgggtttacgacgaaggtgagatagga

SEQ ID NO: 26 - DNA, 22 bp, synthetic construct
  misc_feature 1..22: note = Q_CHK4_ F
  source 1..22: mol_type = other DNA
taagcctgttagagctgctgtg

SEQ ID NO: 27 - DNA, 20 bp, synthetic construct
  misc_feature 1..20: note = Q_CHK4_ R
  source 1..20: mol_type = other DNA
tctttcaaacgcagcagctg

SEQ ID NO: 28 - DNA, 22 bp, synthetic construct
  misc_feature 1..22: note = Q_CHK2_F
  source 1..22: mol_type = other DNA
gctacagaagaacagcgtgttg

SEQ ID NO: 29 - DNA, 20 bp, synthetic construct
  misc_feature 1..20: note = Q_CHK2_R
  source 1..20: mol_type = other DNA
tgtttgctggcaagttggtg

SEQ ID NO: 30 - DNA, 3708 bp, Solanum lycopersicum
  source 1..3708: mol_type = unassigned DNA
atgtccttaaattgcaaattgttgggcatgaatgggagcttctcttcaagttttaggttgaagaaggccagagagtctttgccaggaccaagttgtgggtggaaatggcaaaggaagcttttgtttttatggctcattttctttggaattggatttttttggttggtcattagcttaaatggtgatgtttatagttggaagaaggaggcgtcagaactaaatgaagataaatcttattttcttcttgaacgtttcaatgtcagcaaggaacaaattcaagatttagctaccttattctttgaaaaagatcagatttcatccttggaatgctcaaaagttcataagcacgaaatgcctatgagtactaccattacttgccttctcaaggtgctaggttcggagagtctggaatatgaacagcatgagatggttgtagacaatattgaagcagaaggccagtgcccagttccagatgaggagacactgaaaaatagtgacatttcactagatgaaaagtcgttaccatttgttttgcatcgtttatcttctctaatctcaacagatcctaagttctttgaaaagaaagcatcacaaataagagaagttgggaattttaacccagagcattgtgatagtattgccttctgtttcacaaaactatgctggtgggtccttcttgggattgtgatcagttggaagatacttctgttatgtgcaaaaggtggggaacatcaacagaacggattcattcagcagcaaccactctctcaacaacttcatccactacagcagttgcagcagcagcaagttcagatttcctgtcgaactggtggaaagtggaggaaaaaggctcttgttatctttgtctttggtggggtgactctagccatctggttgtatttgtacctgagtgcagacattgcattgaggaggaaagaaacactgacaagcatgtgtgacgaacgagcgcgaatgttgcaggaccagttcaacgtaagcatgaaccatgttcatgcattggctattcttgtttccacatttcaccatggaaaacaaccttctgcaatagaccagaaaacttttgaagaatatactgagagaacagctttcgaaaggccacttacaagtggtgttgcctatgctttaagagttcgtcactcagagagagaagagtttgagaagctgcatgggtggactataaagaaaatggaagcagaggaccaaactttaggacatgattatatccctgcaaacttggatcctgctcctgatcaagatgaatatgcgcctgtcatattttcacaacaaacagtttcccatatcgtctctattgatatgatgtctggaaaggaagatcgtgagaacattttgcgagcgagggcttctggcaagggggttttgacgtcaccgtttaagctattgaaatccaatcacctgggtgttgttcttacatttgcggtctataatactcatctccttccttatgctaccccggtggaccgcatcaatgctactgttgggtacattggtgcttcttacgatgttccatcattagttgaaaagcttctgcagcagcttgcgagcaaacaaactattgtggtaaatgtttatgatacaactaacaagtttgctccaattaaaatgtatggcatggatgagaacgacacaggattacttcacgttagcaaccttgattttggggatcctgcaaggagccacgagatgcattgcaggttcaagcagaaaccttctccaccctggactgcaataactctatctgtcggagtccttgttatcactctgcttattggtcatatcttccatgctgccatcaacagaattgctgaagttgagggtcagtatcaggaaatgatggagctcaaacatcgtgctgaggctgcagatatagcaaaatcacagtttcttgcaacggtttctcatgaaatcaggactccaatgaatggtgttttaggcatgcttcagatgctcatggacacgaaccttgacctaacgcaactggattatgcacaaactgctcttagtagtgggaatgaactgatatcattgattaatgaggtgctggatcaggctaagattgaatcaggaaggcttgagctggaggctgttccttttgatctccgtgctgaacttgataatgtttcatcgctcttctcgggaaaatctcataaaaaagggattgagttggctgtttatgtttctgacctagtcccagaagttgttatcggagattcaggacggttcaagcagataattacgaatctagttggaaactcagttaagttcacaaatgacaaagggcacatatttgtcacagtacatttagctgatgaagtgaggaaccctcatgatgtgacggatgaagtcttgaaacaaagcttaacctttgttcaagaaaggtcaaatgcatcttggaataccttcagtgggtttcctgtagttgacagatggcaaagttggcaaaagtttgataggctgagcagcacagaggaagaagtgggaaagatcaagttgttagtgaccatagaagacactggtgtgggaattcctcttgaagcacaggcccgcattttcacaccttttatgcaggctgacagttcaacatctcgaacatatggcggaacagggataggattgagcattagcaaacgtttggtagacctcatggggggagaaataggctttttcagtgaacctggcagagggagtacattttcattcactgcagccttcacaagaggagaagaaggttctttagagcgtaaatggaaacagtatgatccagcttttccagaatttcgtgggttaagagcattggtggttgatgataaaagcattagggcagtggtcacaagataccatttgcagagactgggtatatgtgtcaacataacttccacaatgcgttcagcatgctcatatctctctaattactccaatactagttcgttggaacatttagccgtggtttttgttgaccaagatagttgggataaagaaacttctcttgcactaagtaatatgttgaaagaggtcagaccaaatggctcaactactactttggggaagcctccaaaaatctgtctcttatgtatgaactttatggagagagctgagcttaaaatagctggaattgtggatcacgtgttaacaaagcctgtgcggttgagtgcgttgataacatgtgttcaagaagccattggctgccaaaataagaagcaagtgactcaacctacaactcttggaagtttgctgacaggaaagcatatattggtggtagatgacaataatgtaaacaggagagtggcagaaggtgctctaaagaagtatggggcgatagtgacctgtgtagacagtggaaaggctgctttgacacatcttaatccaccacacaagtttgatgcttgttttatggacctccaaatgcctgaaatggatgggtttgaagcgacacgacaaatacgtaatctagaaaacaaatataatgaaaaggtcaattctggtgagttattttccggcatgtctgccagagtggctcattggcacacaccaatattagcaatgacagcagatgtaattcaagcaacaaatgaagagtgcgtgaagtgcggtatggatgattatgtatcaaaaccgtttgaaaaagggcagctttattcaacagtggcacgcttctttgggtcaggttga

SEQ ID NO: 31 - protein, 1235 aa, Solanum lycopersicum
MSLNCKLLGMNGSFSSSFRLKKARESLPGPSCGWKWQRKLLFLWLIFFGIGFFWLVISLNGDVYSWKKEASELNEDKSYFLLERFNVSKEQIQDLATLFFEKDQISSLECSKVHKHEMPMSTTITCLLKVLGSESLEYEQHEMVVDNIEAEGQCPVPDEETLKNSDISLDEKSLPFVLHRLSSLISTDPKFFEKKASQIREVGNFNPEHCDSIAFCFTKLCWWVLLGIVISWKILLLCAKGGEHQQNGFIQQQPLSQQLHPLQQLQQQQVQISCRTGGKWRKKALVIFVFGGVTLAIWLYLYLSADIALRRKETLTSMCDERARMLQDQFNVSMNHVHALAILVSTFHHGKQPSAIDQKTFEEYTERTAFERPLTSGVAYALRVRHSEREEFEKLHGWTIKKMEAEDQTLGHDYIPANLDPAPDQDEYAPVIFSQQTVSHIVSIDMMSGKEDRENILRARASGKGVLTSPFKLLKSNHLGVVLTFAVYNTHLLPYATPVDRINATVGYIGASYDVPSLVEKLLQQLASKQTIVVNVYDTTNKFAPIKMYGMDENDTGLLHVSNLDFGDPARSHEMHCRFKQKPSPPWTAITLSVGVLVITLLIGHIFHAAINRIAEVEGQYQEMMELKHRAEAADIAKSQFLATVSHEIRTPMNGVLGMLQMLMDTNLDLTQLDYAQTALSSGNELISLINEVLDQAKIESGRLELEAVPFDLRAELDNVSSLFSGKSHKKGIELAVYVSDLVPEVVIGDSGRFKQIITNLVGNSVKFTNDKGHIFVTVHLADEVRNPHDVTDEVLKQSLTFVQERSNASWNTFSGFPVVDRWQSWQKFDRLSSTEEEVGKIKLLVTIEDTGVGIPLEAQARIFTPFMQADSSTSRTYGGTGIGLSISKRLVDLMGGEIGFFSEPGRGSTFSFTAAFTRGEEGSLERKWKQYDPAFPEFRGLRALVVDDKSIRAVVTRYHLQRLGICVNITSTMRSACSYLSNYSNTSSLEHLAVVFVDQDSWDKETSLALSNMLKEVRPNGSTTTLGKPPKICLLCMNFMERAELKIAGIVDHVLTKPVRLSALITCVQEAIGCQNKKQVTQPTTLGSLLTGKHILVVDDNNVNRRVAEGALKKYGAIVTCVDSGKAALTHLNPPHKFDACFMDLQMPEMDGFEATRQIRNLENKYNEKVNSGELFSGMSARVAHWHTPILAMTADVIQATNEECVKCGMDDYVSKPFEKGQLYSTVARFFGSG

SEQ ID NO: 32 - DNA, 6097 bp, synthetic construct
  misc_feature 1..6097: note = expression cassette SlyCHK2
  source 1..6097: mol_type = other DNA
ggggacaactttgtatagaaaagttgagtttatcaatattttgagatttttatatatttacattagtaattttcttaataataatctccagaatttataattattttattaattactctaccatcgaatattttttattggttgttctaatttccttgtcactttcaacttgtaattgaccaaaagaaatattttagcttagataagtttcttacatcatatttcctctctctttttatatgctttatttgtataggagtttattattttttatatagtgtttggagcttatattaaatttggatcgcacactgtaaagattcatataaagtgatgcttcgaacaagtttttctctatatctaagctcgaaattatttttctccatacacgaaactcaaatccgaaatatttaattaaagtgaatcagtttcaccccaataccataaccataaatcatgttgattagttgctatcaacttctttcttcatgttcaaatgttttttttaatgaaaaggtattaataattcacatgcattcatcaactagcttaatttaattttattttgataaataattcaataacaatttctactacaattgactcattcaaggattttacatataatgcaaactcacctatatgtatttattaagcacacaatagttgtttagaatcacaagaaatcatataatgatggtcaaactaatcatgtctttttaatattaaaagtgattagttttatctaacactattaaacataccaattcatttattattatattaaataatattattccccttatttttttttttagtaatttaaaacacattagggttattattagggagaagtggggttggagagatgaggtgcattagccagccaactgggatgcccacatcatcaattatcttccaagaataaagtgtaggataagcatctcaagtagccggcgcattttagtacctagcccttgctttattattattttttcacgccgattaatcgccgatcttttgtagttcgtacgccgtattatcaaaagcatagatgctttttttttttaaaaataaatttatttttaaattttaatttatttttgttcttttctccatgtgaaatgaacaaatacaactggctagctcttcccacaaccatcccttttctttttttttctttctttttctcctctcccattctttatatttattatgcctagccatatatttatattttgttcgtttgatttatgtgatgttatttgattagatgttaaaattttaaaatataattatatttataatattttaaattattcttaaaaaaataaattttaaatatttttttatcaaataagtgagagcaaacatgcgatagcgttattaaattctgataagtaagaaaaaatatgaatttaaaaataaacatgtaatataaattagaataaaaaagtattattttttctaatcgaaggcaatatttcgaggggaaaaaccctatatagacctatgtgagatgattttgaattagtaagaatccaatgtgcccgacatcgaatgttttttttttaaaaatatttacagttacattatattgaaactgaaaagggacagtgaaggaatggagctatcttaaaaagcaaagaaaactatctttttattatttttactatatagacaagaaaagaaatatggtaacaaaaatttgcatattgtggaagtgtaaacatttgagaaatgcataattttcggggaaagatgatgatcgaaattttggagatatatcttaattaaattaaggacatattattttaaatatatatttttttgtaattttgtgcatctttttgatgtacgtgacatttaaatattttccacatgcctcaattgcgtgaaattatggagtgtgtcacgtaagacaaaaaatatacaaaataataataaaataatttaagaataataaaacctcaatataattaatgtgtatatatgaaattttaatcataatttaagaggtatttgtatgagcttttctgctctgagtgggtttgctcttaagttttcaaggcattttttgaagatatgtaggtggattctattaaagatgtccttaaattgcaaattgttgggcatgaatgggagcttctcttcaagttttaggttgaagaaggccagagagtctttgccaggaccaagttgtgggtggaaatggcaaaggaagcttttgtttttatggctcattttctttggaattggatttttttggttggtcattagcttaaatggtgatgtttatagttggaagaaggaggcgtcagaactaaatgaagataaatcttattttcttcttgaacgtttcaatgtcagcaaggaacaaattcaagatttagctaccttattctttgaaaaagatcagatttcatccttggaatgctcaaaagttcataagcacgaaatgcctatgagtactaccattacttgccttctcaaggtgctaggttcggagagtctggaatatgaacagcatgagatggttgtagacaatattgaagcagaaggccagtgcccagttccagatgaggagacactgaaaaatagtgacatttcactagatgaaaagtcgttaccatttgttttgcatcgtttatcttctctaatctcaacagatcctaagttctttgaaaagaaagcatcacaaataagagaagttgggaattttaacccagagcattgtgatagtattgccttctgtttcacaaaactatgctggtgggtccttcttgggattgtgatcagttggaagatacttctgttatgtgcaaaaggtggggaacatcaacagaacggattcattcagcagcaaccactctctcaacaacttcatccactacagcagttgcagcagcagcaagttcagatttcctgtcgaactggtggaaagtggaggaaaaaggctcttgttatctttgtctttggtggggtgactctagccatctggttgtatttgtacctgagtgcagacattgcattgaggaggaaagaaacactgacaagcatgtgtgacgaacgagcgcgaatgttgcaggaccagttcaacgtaagcatgaaccatgttcatgcattggctattcttgtttccacatttcaccatggaaaacaaccttctgcaatagaccagaaaacttttgaagaatatactgagagaacagctttcgaaaggccacttacaagtggtgttgcctatgctttaagagttcgtcactcagagagagaagagtttgagaagctgcatgggtggactataaagaaaatggaagcagaggaccaaactttaggacatgattatatccctgcaaacttggatcctgctcctgatcaagatgaatatgcgcctgtcatattttcacaacaaacagtttcccatatcgtctctattgatatgatgtctggaaaggaagatcgtgagaacattttgcgagcgagggcttctggcaagggggttttgacgtcaccgtttaagctattgaaatccaatcacctgggtgttgttcttacatttgcggtctataatactcatctccttccttatgctaccccggtggaccgcatcaatgctactgttgggtacattggtgcttcttacgatgttccatcattagttgaaaagcttctgcagcagcttgcgagcaaacaaactattgtggtaaatgtttatgatacaactaacaagtttgctccaattaaaatgtatggcatggatgagaacgacacaggattacttcacgttagcaaccttgattttggggatcctgcaaggagccacgagatgcattgcaggttcaagcagaaaccttctccaccctggactgcaataactctatctgtcggagtccttgttatcactctgcttattggtcatatcttccatgctgccatcaacagaattgctgaagttgagggtcagtatcaggaaatgatggagctcaaacatcgtgctgaggctgcagatatagcaaaatcacagtttcttgcaacggtttctcatgaaatcaggactccaatgaatggtgttttaggcatgcttcagatgctcatggacacgaaccttgacctaacgcaactggattatgcacaaactgctcttagtagtgggaatgaactgatatcattgattaatgaggtgctggatcaggctaagattgaatcaggaaggcttgagctggaggctgttccttttgatctccgtgctgaacttgataatgtttcatcgctcttctcgggaaaatctcataaaaaagggattgagcagttggctgtttatgtttctgacctagtcccagaagttgttatcggagattcaggacggttcaagcagataattacgaatctagttggaaactcagttaagttcacaaatgacaaagggcacatatttgtcacagtacatttagctgatgaagtgaggaaccctcatgatgtgacggatgaagtcttgaaacaaagcttaacctttgttcaagaaaggtcaaatgcatcttggaataccttcagtgggtttcctgtagttgacagatggcaaagttggcaaaagtttgataggctgagcagcacagaggaagaagtgggaaagatcaagttgttagtgaccatagaagacactggtgtgggaattcctcttgaagcacaggcccgcattttcacaccttttatgcaggctgacagttcaacatctcgaacatatggcggaacagggataggattgagcattagcaaacgtttggtagacctcatggggggagaaataggctttttcagtgaacctggcagagggagtacattttcattcactgcagccttcacaagaggagaagaaggttctttagagcgtaaatggaaacagtatgatccagcttttccagaatttcgtgggttaagagcattggtggttgatgataaaagcattagggcagtggtcacaagataccatttgcagagactgggtatatgtgtcaacataacttccacaatgcgttcagcatgctcatatctctctaattactccaatactagttcgttggaacatttagccgtggtttttgttgaccaagatagttgggataaagaaacttctcttgcactaagtaatatgttgaaagaggtcagaccaaatggctcaactactactttggggaagcctccaaaaatctgtctcttatgtatgaactttatggagagagctgagcttaaaatagctggaattgtggatcacgtgttaacaaagcctgtgcggttgagtgcgttgataacatgtgttcaagaagccattggctgccaaaataagaagcaagtgactcaacctacaactcttggaagtttgctgacaggaaagcatatattggtggtagatgacaataatgtaaacaggagagtggcagaaggtgctctaaagaagtatggggcgatagtgacctgtgtagacagtggaaaggctgctttgacacatcttaatccaccacacaagtttgatgcttgttttatggacctccaaatgcctgaaatggatgggtttgaagcgacacgacaaatacgtaatctagaaaacaaatataatgaaaaggtcaattctggtgagttattttccggcatgtctgccagagtggctcattggcacacaccaatattagcaatgacagcagatgtaattcaagcaacaaatgaagagtgcgtgaagtgcggtatggatgattatgtatcaaaaccgtttgaaaaagggcagctttattcaacagtggcacgcttctttgggtcaggttgagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggacaactttgtataataaagttg

(19) United States
(12) Patent Application Publication
(10) Pub. No.: US 2025/0369007 A1
(43) Pub. Date: Dec. 4, 2025

(54) SHOOT REGENERATION BY OVEREXPRESSION OF CHK GENES

(71) Applicant: Keygene N.V., Wageningen (NL)

(72) Inventors: Michiel Theodoor Jan DE BOTH, Wageningen (NL); Rik Hubertus Martinus OP DEN CAMP, Wageningen (NL); Bjorn Alexander KLOOSTERMAN, Wageningen (NL); Bernarda Gerharda Johanna FIERENS-ONSTENK, Wageningen (NL)

(73) Assignee: Keygene N.V., Wageningen (NL)

(21) Appl. No.: 19/296,788

(22) Filed: Aug. 11, 2025

Related U.S. Application Data
(63) Continuation of application No. 17/062,221, filed on Oct. 2, 2020, now Pat. No. 12,398,399.
Application No. 17/062,221 is a continuation of application No. PCT/EP2019/058614, filed on Apr. 5, 2019.

(30) Foreign Application Priority Data
Apr. 5, 2018 (EP) 18165895.6

(51) Int. Cl. C12N 15/82

(52) U.S. Cl. CPC C12N 15/8262; C12N 15/8295

(57) ABSTRACT

Genetic modification of plants is hampered by the limited capacity of plant cells to regenerate. The current invention solves this problem by introducing or increasing the expression of a histidine kinase in a plant cell. Preferred histidine kinases are at least one of CHK2, CHK3 and CHK4. The invention therefore concerns a method for improving a cytokinin-induced regeneration capacity of a plant cell, wherein the method comprises a step of increasing or introducing the expression of a histidine kinase in the plant cell. The invention further pertains to a method for regenerating a plant, wherein the method comprises a step of introducing or increasing the expression of a histidine kinase and to a plant obtainable from such method. Moreover, the method concerns the use of at least one of CHK2, CHK3 and CHK4 for improving a cytokinin-induced regeneration capacity of a plant.

### CROSS-REFERENCE TO RELATED APPLICATIONS

This application is a continuation of U.S. application Ser. No. 17/062,221 filed Oct. 2, 2020, which is a Bypass continuation of International Application No. PCT/EP2019/058614 filed Apr. 5, 2019, which claims the benefit of and priority to European Application No. 18165895.6 filed Apr. 5, 2018, both of which are hereby incorporated by reference herein in their entireties.

### SEQUENCE LISTING

The instant application contains a Sequence Listing which has been submitted electronically in XML format and is hereby incorporated by reference in its entirety. Said XML copy, created on Aug. 18, 2025, is named 085342-3801_SL.xml and is 81,716 bytes in size.

### FIELD OF THE INVENTION

The present invention relates to the field of molecular plant biology, in particular to the field of plant regeneration. The invention concerns methods for improving the regeneration capacity of plant cells.

### BACKGROUND

Plant breeding aims to improve the productivity and performance of crop plants through selection and recombination of useful and superior traits, and to improve such plant traits by genetic technologies. These technologies are often faced with a central technical challenge: how to regenerate from a single cell back to a fertile plant. This is true both for techniques aimed at making genetic changes (mutagenesis, genome editing, genetic transformation) and for those affecting the genome as a whole (DH production, polyploidisation, somatic hybridisations). This is because the genetic or genomic changes realised occur in only one or a small number of cells, and never in all cells of an organism at once in exactly the same manner. Therefore, an important limitation of these current techniques is the identification and singling out of the desired cells, and subsequently growing them back to complete and fertile plants.

Techniques of genome improvement are hampered by the potential of in vitro regeneration of modified single cells into whole fertile plants. Regeneration typically passes through a stage whereby a single cell first undergoes sustained cell division to form a multicellular structure or callus. Subsequently, under the influence of exogenously supplied plant growth regulators, cells in the multicellular mass form organized structures. Hence regeneration involves at least two sequential critical steps, i.e. a step of cell division followed by a step of differentiation to form organized structures. The first step one may involve callus formation. The second step must involve de novo meristem formation.

In vitro plant regeneration follows one of two alternative pathways, organogenesis and somatic embryogenesis, both of which rely on the induction by plant growth regulators (Duclercq et al., 2011, TIPS 16:597). Monocotyledonous and dicotyledonous plants can use either both or only one of these two alternative pathways to regenerate.

A common pathway of plant regeneration in dicotyledonous plants is organogenesis, in which de novo apical meristems are formed from undifferentiated cells. These meristems usually grow out to form shoots, which are then dissected from the underlying cell mass and induced to form roots. Organogenesis is typically induced in culture media containing cytokinins, or a mixture of auxins or cytokinins in which the cytokinins are often predominantly present. Cytokinins are a group of plant growth regulators or phytohormones, derivatives of adenine, and capable of promoting cell division (Mok and Mok, 2001, Annu. Rev. Plant Physiol. Plant Mol. Biol. 52: 89-118). The group encompasses naturally occurring cytokinins such as zeatin, and synthetic cytokinins such as kinetin and 6-benzylaminopurine (6-BAP).

Another regeneration pathway, often found in monocotyledonous and woody species, is through somatic embryogenesis, whereby undifferentiated cells in a callus, under proper conditions, generate embryogenic cells that ultimately form structures that resemble zygotic embryos, the so-called somatic embryos (Quiroz-Figueroa et al., 2006, PCTOC 86: 285-301). These somatic embryos are then capable of being converted into small plantlets, either spontaneous or under low concentrations of plant growth regulators. Embryogenic potential in callus or cell suspensions is typically induced by auxins, a group of naturally occurring or synthetic plant growth regulators.

A major bottleneck is the generally limited capacity of plant cells to regenerate into fertile and healthy plants. Regeneration potential is highly dependent on plant species, on variety and on tissue origin. Even with established protocols, the fraction of cells successfully regenerating to plants is usually quite low (Srinivasan et al., 2007, Planta 225: 341-351). Plant species or varieties in which the regeneration fails or the efficiency is poor are considered recalcitrant. Examples of crop species recalcitrant in organogenesis are pepper, soybean, cucumber and sugar beet.

In the past, recalcitrance has often been addressed by trial and error approaches through empirical variations in tissue culture conditions (media composition, light, temperature), and met with limited success. Protocols developed in this way are still dependent on genotype and laboratory conditions, and therefore to some extent unpredictable.

Regeneration through organogenesis or somatic embryogenesis can be enhanced by the ectopic expression of transgenes. An example of such approach is the overexpression of ESR1 (an AP2/EREBP transcription factor) in Arabidopsis (Banno et al., 2001, Plant Cell 13: 2609-2618), resulting in enhanced shoot regeneration from root explants. Another example is the overexpression of BBM, an AP2/ERF transcription factor, in tobacco (Srinivasan et al., 2007, Planta 225: 341-351), resulting in increased shoot formation and a higher competence for somatic embryogenesis. However, the constitutive expression of these transcription factors results in morphological and developmental defects, and these therefore require the controlled expression by exogenously supplied inducers (estradiol-inducible and dexamethasone-inducible system, respectively). The requirement of controlled expression thus currently limits the use of transgenes for regeneration.

Hence, there is still a need in the art for improving the generation capacity of a plant, especially for improving the generation capacity of a plant having a low or insufficient regeneration capacity. In particular, there is also a need in the art to improve the regeneration capacity of a plant, without requiring the controlled expression of transgenes.

### SUMMARY OF THE INVENTION

In a first aspect, the invention pertains to a method for improving a cytokinin-induced regeneration capacity of a plant cell, wherein the method comprises a step of increasing or introducing the expression of a histidine kinase in the plant cell and wherein the histidine kinase is at least one of CHK2, CHK3 and CHK4, wherein preferably the histidine kinase is at least one of CHK2 and CHK4, preferably wherein the histidine kinase is CHK4.

In one embodiment, the histidine kinase is encoded by a nucleotide sequence having at least 50% sequence identity with at least one of SEQ ID NO: 1, SEQ ID NO:2 and SEQ ID NO: 3, wherein preferably the nucleotide sequence has at least 50% sequence identity with SEQ ID NO: 3.

Preferably, the regeneration capacity of the plant cell is improved as compared to an identical plant cell not having an increased or introduced expression of the histidine kinase.

In an embodiment, the amino acid sequence of the histidine kinase has at least 50% sequence identity with at least one of SEQ ID NO: 4, SEQ ID NO: 5 and SEQ ID NO: 6, wherein preferably the amino acid sequence of the histidine kinase has at least 50% sequence identity with SEQ ID NO: 6

Preferably, the expression of the histidine kinase is transiently increased or introduced into the plant cell.

In one embodiment of the invention, the expression of the histidine kinase is continuously increased or introduced in the plant cell.

Preferably, the plant cell is obtainable from a plant selected from the group consisting of barley, cabbage, canola, cassava, cauliflower, chicory, cotton, cucumber, eggplant, grape, hot pepper, lettuce, maize, melon, oilseed rape, potato, pumpkin, rice, rye, sorghum, squash, sugar cane, sugar beet, sunflower, sweet pepper, tomato, water melon, wheat, zucchini, soybean, chrysanthemum and Arabidopsis.

In a further embodiment, the cytokinin is an adenine-type cytokinin, wherein preferably the adenine-type cytokinin is selected from the group consisting of kinetin, zeatin, trans-zeatin, cis-zeatin, dihydrozeatin, 6-benzylaminopurine and 2iP.

In a second aspect, the invention concerns a method for regenerating a plant comprising the steps of:
incubating the plant cell as defined herein in a medium comprising a cytokinin; and
allowing the plant cell to regenerate into a plant.

Preferably the medium comprises at least one further plant hormone, wherein preferably the one further plant hormone is an auxin.

In an embodiment, the plant cell is part of a multicellular tissue, preferably a callus tissue, a plant organ or an explant.

Preferably the explant is at least one of a hypocotyl explant, a stem explant, a cotyledon explant, a root explant, a leaf explant, a flower explant and a meristematic tissue.

In a further embodiment, the concentration cytokinin in the medium is about 100-3000 ng/ml, wherein preferably the concentration cytokinin is about 200-600 ng/ml.

In an embodiment of the invention, the improved cytokinin-induced regeneration capacity is selected from the group of an improved meristem formation, an improved adventitious shoot formation, an improved inflorescence formation, an improved somatic embryo formation, an improved root formation, an improved elongation of adventitious shoots and an improved regeneration of a complete plant.

In a third aspect, the invention relates to a plant or plant part obtainable by the method of the invention as defined herein, wherein preferably the plant part is a seed, a fruit or a non-propagating material.

In another aspect, the invention pertains to an expression construct comprising a first nucleotide sequence having at least 50% sequence identity with SEQ ID NO: 3 and a second nucleotide sequence having at least 50% sequence identity with SEQ ID NO: 1, wherein preferably at least one of the first and the second nucleotide sequence is operably linked to a regulatory element.

In a further aspect, the invention concerns the use of a CHK2, CHK3 and/or CHK4 histidine kinase or the expression construct as defined herein, for improving a cytokinin-induced regeneration capacity of a plant.

### DEFINITIONS

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein.

It is clear for the skilled person that any methods and materials similar or equivalent to those described herein can be used for practising the present invention.

Methods of carrying out the conventional techniques used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al. Molecular Cloning. A Laboratory Manual, 2nd Edition, Cold Spring Harbor Laboratory Press, Cold Spring Harbor, N.Y., 1989; Ausubel et al. Current Protocols in Molecular Biology, John Wiley & Sons, New York, 1987 and periodic updates; and the series Methods in Enzymology, Academic Press, San Diego.

The singular terms “a,” “an,” and “the” include plural referents unless the context clearly dictates otherwise. Thus, for example, reference to “a cell” includes a combination of two or more cells, and the like. The indefinite article “a” or “an” thus usually means “at least one”.

The term “and/or” refers to a situation wherein one or more of the stated cases may occur, alone or in combination with at least one of the stated cases, up to with all of the stated cases.

As used herein, the term “about” is used to describe and account for small variations. For example, the term can refer to less than or equal to ± (+or −) 10%, such as less than or equal to ±5%, less than or equal to ±4%, less than or equal to ±3%, less than or equal to ±2%, less than or equal to +1%, less than or equal to ±0.5%, less than or equal to ±0.1%, or less than or equal to ±0.05%. Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “comprising” is construed as being inclusive and open ended, and not exclusive. Specifically, the term and variations thereof mean the specified features, steps or components are included. These terms are not to be interpreted to exclude the presence of other features, steps or components.

The terms “protein” or “polypeptide” are used interchangeably and refer to molecules consisting of a chain of amino acids, without reference to a specific mode of action, size, 3 dimensional structure or origin. A “fragment” or “portion” of a protein may thus still be referred to as a “protein.” An “isolated protein” is used to refer to a protein which is no longer in its natural environment, for example in vitro or in a recombinant bacterial or plant host cell.

“Plant” refers to either the whole plant or to parts of a plant, such as cells, tissue or organs (e.g. pollen, seeds, gametes, roots, leaves, flowers, flower buds, anthers, fruit, etc.) obtainable from the plant, as well as derivatives of any of these and progeny derived from such a plant by selfing or crossing.

“Plant cell(s)” include protoplasts, gametes, suspension cultures, microspores, pollen grains, etc., either in isolation or within a tissue, organ or organism. The plant cell can e.g. be part of a multicellular structure, such as a callus, meristem, plant organ or an explant.

“Similar conditions” for culturing the plant/plant cells means among other things the use of a similar temperature, humidity, nutrition and light conditions, and similar irrigation and day/night rhythm.

The terms “homology”, “sequence identity” and the like are used interchangeably herein. Sequence identity is herein defined as a relationship between two or more amino acid (polypeptide or protein) sequences or two or more nucleotide (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods. The percentage sequence identity/similarity can be determined over the full length of the sequence.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithms (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall lengths, local alignments, such as those using the Smith Waterman algorithm, are preferred.

Alternatively percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

The terms “nucleic acid construct”, “nucleic acid vector”, “vector” and “expression construct” are used interchangeably herein and is herein defined as a man-made nucleic acid molecule resulting from the use of recombinant DNA technology. The terms “nucleic acid construct” and “nucleic acid vector” therefore does not include naturally occurring nucleic acid molecules although a nucleic acid construct may comprise (parts of) naturally occurring nucleic acid molecules.

The vector backbone may for example be a binary or superbinary vector (see e.g. U.S. Pat. No. 5,591,616, US 2002138879 and WO 95/06722), a co-integrate vector or a T-DNA vector, as known in the art and as described elsewhere herein, into which a chimeric gene is integrated or, if a suitable transcription regulatory sequence is already present, only a desired nucleic acid sequence (e.g. a coding sequence, an antisense or an inverted repeat sequence) is integrated downstream of the transcription regulatory sequence. Vectors can comprise further genetic elements to facilitate their use in molecular cloning, such as e.g. selectable markers, multiple cloning sites and the like.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′ non-translated sequence (3′ end) comprising a polyadenylation site.

“Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

The term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked may mean that the DNA sequences being linked are contiguous.

“Promoter” refers to a nucleic acid fragment that functions to control the transcription of one or more nucleic acids. A promoter fragment is preferably located upstream (5′) with respect to the direction of transcription of the transcription initiation site of the gene, and is structurally identified by the presence of a binding site for DNA-dependent RNA polymerase, transcription initiation site(s) and can further comprise any other DNA sequences, including, but not limited to transcription factor binding sites, repressor and activator protein binding sites, and any other sequences of nucleotides known to one of skill in the art to act directly or indirectly to regulate the amount of transcription from the promoter.

Optionally the term “promoter” may also include the 5′ UTR region (5′ Untranslated Region) (e.g. the promoter may herein include one or more parts upstream of the translation initiation codon of transcribed region, as this region may have a role in regulating transcription and/or translation). A “constitutive” promoter is a promoter that is active in most tissues under most physiological and developmental conditions. An “inducible” promoter is a promoter that is physiologically (e.g. by external application of certain compounds) or developmentally regulated. A “tissue specific” promoter is only active in specific types of tissues or cells.

The term “regeneration” is herein defined as the formation of a new tissue and/or a new organ from a single plant cell, a callus, an explant, a tissue or from an organ. A preferred regeneration pathway is organogenesis, i.e. the formation of new organs from (undifferentiated) cells. Preferably, the regeneration is at least one of ectopic apical meristem formation, shoot regeneration and root regeneration. The regeneration as defined herein can preferably concern at least de novo shoot formation. For example, regeneration can be the regeneration of a(n) (elongated) hypocotyl explant towards a(n) (inflorescence) shoot.

Regeneration may further include the formation of a new plant from a single plant cell or from e.g. a callus, an explant, a tissue or an organ. The regeneration process can occur directly from parental tissues or indirectly, e.g. via the formation of a callus.

The term “conditions that allow for regeneration” is herein understood as an environment wherein a plant cell or tissue can regenerate. Such conditions include at minimum a suitable temperature (i.e. between 0° C.-60° C.), nutrition, day/night rhythm, irrigation and the plant hormone cytokinin. These conditions can include one, two or three of the conditions specified in the examples. Furthermore, “optimal conditions that allow for regeneration” are those environmental conditions that allow for a maximum regeneration of the plant cells.

The terms “introduced expression” and “de novo expression” are used interchangeable herein.

### DETAILED DESCRIPTION OF THE INVENTION

The inventors discovered that introducing or enhancing the expression of a histidine kinase in a plant cell increases the regeneration capacity of this plant cell when grown in a medium comprising a cytokinin. The regeneration potential surprisingly increased in comparison to unmodified plant cells grown in the same cytokinin-comprising medium. Introducing a histidine kinase in a plant cell thus significantly augments the effect of the cytokinin. This enhanced effect of increasing the expression of a histidine kinase in combination with growing the cells on cytokinin-comprising medium surprisingly increased the maximum ability of a cell to regenerate. The inventors thus discovered that introducing a histidine kinase into a plant cell can transform a plant cell having a poor regeneration capacity into a plant cell that can regenerate, when grown in a cytokinin-comprising medium.

Without wishing to be bound to any theory, histidine kinase genes such as CHK genes encoding the cytokinin receptor molecules, seem to render plants more sensitive, and consequently more responsive to cytokinin, resulting in greatly improved shoot regeneration capacity. CHK genes previously have been implicated in the cytokinin signal transduction (Kakimoto, 1996, Science 274: 982-985), and enhanced expression of CHK genes have been associated with functions attributed to cytokinin response, but not regeneration capacity (Deng et al., 2010, Plant Cell 22: 1232-1248).

WO02/099079, EP2272862, and Riefler M et al, (The Plant Cell, Vol. 18 (2006): p40-54) concern cytokinin response regulators and uses thereof. These documents are however silent on the combination of expressing a histidine kinase and maintaining the cells in cytokinin-comprising medium. In particular, the inventors now show that expressing a histidine kinase in cells in combination with maintaining the cells in cytokinin-comprising medium can push the cells above their natural maximum ability to regenerate.

In a first aspect, the invention therefore pertains to a method for improving a cytokinin-induced regeneration capacity of a plant cell, wherein the method comprises a step of increasing or introducing the expression of a histidine kinase in the plant cell.

The plant cell may be a protoplast. A cytokinin-induced regeneration is defined herein as the regeneration of a plant cell when exposed to at least a cytokinin, e.g. the regeneration response of a plant cell when exposed to a cytokinin under conditions that allow for the regeneration of the plant cell.

The cytokinin-induced regeneration capacity of a plant cell is herein understood as the maximum potential of a plant cell to regenerate when exposed to at least the plant hormone cytokinin under conditions, preferably optimal conditions, that allow for the regeneration of the plant cell. It is well-known in the art that plant cells have a certain inherent maximum potential to regenerate in a medium comprising a cytokinin. For example, the maximum regeneration potential of a plant cell obtained from e.g. a tobacco plant is usually higher than the maximum regeneration potential of a plant cell obtained from e.g. a cucumber plant. The regeneration capacity of plant cells may be assessed by determining the number of de novo formed shoots on a multicellular tissue or tissues. As a non-limiting example upon induction of regeneration, tobacco multicellular tissues may provide for a higher number of de novo formed shoots per multicellular tissue as compared to the same number of cucumber multicellular tissues. A preferred multicellular tissue is a callus or explant.

In addition or alternatively, the maximum potential of a plant cell to regenerate can for example be measured by comparing the amount of starting material with the amount of material that is regenerated after exposure to cytokinin. As a non-limiting example, the number of multicellular tissues that form the starting material can be determined. Subsequently after exposure to at least a cytokinin under conditions, preferably optimal conditions that allow for regeneration, the number of multicellular tissues that are regenerated can be determined. The percentage of regenerated multicellular tissues is herein understood to be the cytokinin-induced regeneration capacity of the plant cell. As non-limiting example, the starting material can be a hypocotyl explant, such as an elongated hypocotyl explant, and/or the regenerated tissue can be a shoot, such as an inflorescence shoot.

The cytokinin-induced regeneration may be at least one of meristem formation, adventitious shoot formation, inflorescence formation, somatic embryo formation, root formation, elongation of adventitious shoots and regeneration of a complete plant. As a non-limiting example, the number of de novo formed shoots per multicellular tissue may be determined.

The skilled person understands that there are other similar approaches known in the art to determine the cytokinin-induced regeneration capacity.

Preferably, the cytokinin-induced regeneration capacity of the plant cell is improved as compared to a control preferably as tested under the same, or substantially the same conditions, or optimal conditions. Preferably, said control is an identical plant cell or substantial identical plant cell that has the same genetic background as compared to the plant cell obtained or obtainable by the method of the invention, with the exception of the modified or newly introduced histidine kinase. In other words, preferably, the control is a plant cell that is genetically identical to the plant cell obtained by the method of the invention before the step of increasing or introducing the expression of a histidine kinase of the method of the invention. Thus, preferably the identical plant cell or substantially identical plant cell has the same genetic background as the plant cell having an improved regeneration capacity, but does not have an introduced or increased expression of the histidine kinase as defined herein.

The cytokinin-induced regeneration capacity of a plant cell is the maximum potential of the plant cell to regenerate under conditions, preferably optimal conditions, that allow for regeneration. The optimal conditions preferably includes at least an optimal concentration of a cytokinin.

The regeneration capacity can be expressed as the percentage of the starting material that regenerates. As a non-limiting example, if 7 of the 360 explants regenerate under conditions that allow for regeneration, the regeneration capacity is (7/360*100%) 1.9%. Expression of a histidine kinase as defined herein allows for an improved regeneration capacity. As a non-limiting example, introduced or increased expression of a histidine kinase can increase the number of regenerated explants to e.g. 160 when grown under similar or substantially the same regeneration conditions. The regeneration capacity has increased to (160/360*100%) to 44.4%. Hence, the regeneration capacity has improved (44.4−1.9) 42.5% (see e.g. Table 2). As a further non-limiting example, the regeneration capacity of a plant cell can be expressed as the number of shoots formed per explant at a certain time point. For example 183 days after induced regeneration, 93 shoots can be harvested from 200 explants that have an introduced or increased expression of a histidine kinase (see also table 4), e.g. the regeneration capacity at day 183 is (93/200*100%) 46.5%. At the same time point 64 shoots can be harvested from 200 control explants, e.g. the regeneration capacity at day 183 is (64/200*100%) 32%. The regeneration capacity thus has improved ((46.5−32)/32*100%) 45% (see e.g. Table 4).

As a non-limiting example, the improvement in cytokinin-induced regeneration capacity can be expressed as the difference in regeneration capacity between a plant cell having an increased or introduced expression of the histidine kinase as defined herein and an identical cell not having said increased or introduced expression. Preferably, the regeneration capacity of the plant cell is improved at least about 1%, 2%, 5%, 10%, 15%, 20%, 25%, 30%, 35%, 40%, 45%, 50%, 55%, 60%, 65%, 70%, 75%, 80%, 85%, 90%, 95%, or about 100% as compared to an identical plant cell not having an increased or introduced expression of the histidine kinase.

The skilled person understands that there are other suitable approaches in the art to determine an improvement in cytokinin-induced regeneration capacity, which can be equally used in the method of the invention.

A regenerated plant cell has preferably at least one of meristem formation or de novo meristem formation, shoot formation or adventitious shoot formation, inflorescence formation, somatic embryo formation, root formation, elongation of adventitious shoots and the regeneration of a complete plant.

The method can further comprise, for example, testing the plant cell for an improved regeneration capacity. The improved regeneration capacity is preferably improved compared to a control, wherein the control is preferably an identical plant cell not having an increased or introduced expression of a histidine kinase as defined herein.

As a non-limiting example, the testing step may be performed by determining the number of de novo formed shoots. Hence, the method of the invention may further comprise a step of determining, e.g. counting, the number of de novo formed shoots. The number of de novo formed shoots can be determined in relation to the number of multicellular tissues, preferably the number of explants, prior to shoot formation. Alternatively or in addition, the number of meristems may be determined or any tissues derived therefrom. It is however understood herein that the formation of callus or “green callus” is not indicative of the number of shoots that can be formed. The formation of callus can be an indicator of proliferation, but not an indicator of organisation/differentiation.

As a further non-limiting example, the testing step may be performed by determining the number of explants that regenerate. Hence, the method of the invention may further comprise a step of determining, e.g. counting, the number of explants that have regenerated. The number of regenerated explants can be determined in relation to the number of explants that formed the starting material.

A histidine kinase for use in the invention is a protein that is capable of transferring a phosphate group to a histidine residue on a specific substrate. Preferably, the histidine kinase for use in the invention is a transmembrane protein and can act as a cellular receptor for cytokinin. Upon binding the cytokinin, the histidine kinase can initiate a signal transduction resulting in the activation of Type B ARRs (Arabidopsis response regulators) and subsequent cytokinin-regulated transcription.

The histidine kinase may be a CHASE-domain containing histidine kinase (CHK). These receptors preferably display a complex multidomain structure with a N-terminal part including preferably at least two hydrophobic membrane-spanning domains (TM) that border an extracytosolic sensing domain referred to as CHASE (Cyclase/Histidine kinase Associated Sensory Extracellular) as well as a cytoplasmic C-terminal part containing a catalytic histidine kinase (HK) domain and both receiver and pseudo-receiver domains (REC and REC-like, respectively). The HK domain is preferably composed of an HK dimerization and phosphoacceptor domain (HisKA) and an HK catalytic domain called the HK-like ATPase domain (HATPase) (Daudu et al, “CHASE-Containing Histidine Kinase Receptors in Apple Tree: From a Common Receptor Structure to Divergent Cytokinin Binding Properties and Specific Functions”, Front Plant Sci. (2017); 8: 1614).

The histidine kinase for use in the invention can be a protein that is native to the plant cell, e.g. an endogenous histidine kinase protein that is expressed, or overexpressed, in the cell. Hence, the endogenous protein can be a protein that is encoded in the genome of a wild-type plant cell and its expression is introduced or enhanced. In addition or alternatively, additional copies of the endogenously encoded protein can be introduced into the plant cell.

In an embodiment, the histidine kinase for use in the invention is not native, i.e. is foreign, to the plant cell. Such exogenous protein may be a homologous protein. The protein can be derived from the same subgenus, genus, tribe, subfamily, family, order and/or clade. Alternatively, the protein can be derived from a different subgenus, genus, tribe, subfamily, family, order and/or clade. The histidine kinase protein can be an artificial protein, e.g. a protein that does not occur in nature but fulfils the same or similar function as a naturally-occurring histidine kinase.

In a preferred embodiment, the histidine kinase is at least one of CHK2, CHK3 and CHK4. Preferably the histidine kinase is at least one of CHK2 and CHK4. In an embodiment, the histidine kinase is CHK2. In another embodiment, the histidine kinase is CHK4. It is envisioned herein that the expression of a combination of histidine kinases is introduced or increased in the plant cell. For example, the expression of at least two different CHK2 proteins, at least two different CHK3 and/or at least two different CHK4 proteins is increased or introduced in the plant cell. Alternatively or in addition, the plant cell may have an increased or introduced expression of at least a CHK2 and CHK4 protein, at least a CHK2 and CHK3 protein, or at least a CHK3 and CHK4 protein. The plant cell can have an increased or introduced expression of at least a CHK2, CHK3 and a CHK4 protein as defined herein.

The histidine kinase of the invention may be encoded by a nucleotide sequence having at least 50% sequence identity with at least one of SEQ ID NO: 1, SEQ ID NO:2 and SEQ ID NO: 3. Preferably, the nucleotide sequence encoding the CHK2 can have at least about 50%, 55%, 60%, 65%, 70%, 75%, 80%, 85%, 90%, 95%, 98%, 99% or 100% sequence identity with SEQ ID NO: 1. SEQ ID NO: 1 is the Abidopsis CHK2 coding sequence, also annotated as AHK2.

Preferably, the nucleotide sequence encoding the CHK3 can have at least about 50%, 55%, 60%, 65%, 70%, 75%, 80%, 85%, 90%, 95%, 98%, 99% or 100% sequence identity with SEQ ID NO: 2. SEQ ID NO: 2 is the Arabidopsis CHK3 coding sequence, also annotated as AHK3.

Preferably, the nucleotide sequence encoding the CHK4 preferably has at least about 50%, 55%, 60%, 65%, 70%, 75%, 80%, 85%, 90%, 95%, 98%, 99% or 100% sequence identity with SEQ ID NO: 3. SEQ ID NO: 3 is the Arabidopsis CHK4 coding sequence, also annotated as AHK4, CRE1, WOL1, WOODEN LEG 1.

In one embodiment, the nucleotide sequence encoding the CHK2 protein is, or is derived from the gene At5g35750 (SEQ ID NO: 7), a homolog thereof or a sequence having at least about 50%, 55%, 60%, 65%, 70%, 75%, 80%, 85%, 90%, 95%, 98%, 99% or 100% sequence identity with At5g35750 or its homolog. The percentage identity can be determined over the full length of the genomic sequence. Alternatively the percentage identity can be determined over the full length of the coding sequence of the gene.

Examples of homologs include Glycine max (Soybean) (GLYMA02G47611 or GLYMA14G01040), Oryza sativa (Rice) (OS10G0362300, OSJNBA0058E19.1 or OSJNBA0073L01.1), Populus trichocarpa (Black Cottonwood) (POPTR_0014S16260G), Solanum lycopersicum (Tomato) (SOLYC07G047770.2) and Vitis vinifera (Grape) (VIT_12S0057G00690). A preferred homolog is Solanum lycopersicum SOLYC07G047770.2.

Preferably, the nucleotide sequence encoding the CHK2 protein is, or is derived from the gene SOLYC07G047770.2 (SEQ ID NO: 30), a homolog thereof and/or a sequence having at least about 50%, 55%, 60%, 65%, 70%, 75%, 80%, 85%, 90%, 95%, 98%, 99% or 100% sequence identity with SOLYC07G047770.2. The percentage identity can be determined over the full length of the genomic sequence. Alternatively the percentage identity can be determined over the full length of the coding sequence of the gene.

In one embodiment, the sequence encoding the CHK3 protein is, or is derived from the gene At1g27320 (SEQ ID NO: 8), a homolog thereof or a sequence having at least about 50%, 55%, 60%, 65%, 70%, 75%, 80%, 85%, 90%, 95%, 98%, 99% or 100% sequence identity with At1g27320 or its homolog. The percentage identity can be determined over the full length of the genomic sequence. Alternatively the percentage identity can be determined over the full length of the coding sequence of the gene.

Examples of homologs include Brachypodium distachyon (Purple false brome) (BRADI2G59127), Glycine max (Soybean) (GLYMA05G28070 or GLYMA08G11060), Oryza sativa (Rice) (B1455F06.33), Populus trichocarpa (Black Cottonwood) (HK3A or POPTR_0003S16950G), Solanum lycopersicum (Tomato) (SOLYC05G015610.2) and Vitis vinifera (Grape) (VIT_01S0010G03780).

In one embodiment, the sequence encoding the CHK4 protein is, or is derived from, the gene At2g01830 (SEQ ID NO: 9), a homolog thereof, or a sequence having at least about 50%, 55%, 60%, 65%, 70%, 75%, 80%, 85%, 90%, 95%, 98%, 99% or 100% sequence identity with At2g01830 or its homolog. The percentage identity can be determined over the full length of the genomic sequence. Alternatively the percentage identity can be determined over the full length of the coding sequence of the gene.

Examples of homologs include Brachypodium distachyon (Purple false brome) (BRADI1G10660), Glycine max (Soybean) (GLYMA02G09550, GLYMA05G34310, GLYMA07G19620, GLYMA07G27540 or GLYMA08G05370), Physcomitrella patens (Moss) (CKI3A, CKI3B, CKI3C, CRE1, CRE2, CRE3, PHYPADRAFT_162473, PHYPADRAFT_169293, PHYPADRAFT_172225, PHYPADRAFT_229095 or PHYPADRAFT_68053), Populus trichocarpa (Black Cottonwood) (CRE1B, POPTR_0008S13720G), Solanum lycopersicum (Tomato) (SOLYC04G008110.2), Sorghum bicolor (Sorghum) (SB01G010070) and Vitis vinifera (Grape) (VIT_01S0011G06190)

The sequence encoding the histidine kinase may be codon-optimized for expression in plant cells, preferably codon-optimized for expression in the plant cell of the method of the invention. As a non-limiting example, the expressed or de novo expressed histidine kinase can be an endogenous protein while the sequence encoding this endogenous protein is an exogenous, codon-optimized, sequence. Alternatively, the codon-optimized sequence can encode a histidine kinase that is exogenous for the plant cell.

In one embodiment, the amino acid sequence of the histidine kinase has at least 50% sequence identity with at least one of SEQ ID NO: 4, SEQ ID NO: 5 and SEQ ID NO: 6.

The amino acid sequence encoding the CHK2 can have at least about 50%, 55%, 60%, 65%, 70%, 75%, 80%, 85%, 90%, 95%, 98%, 99% or 100% sequence identity with SEQ ID NO: 4. SEQ ID NO: 4 is the Arabidopsis CHK2 protein.

Alternatively, the nucleotide sequence encoding the CHK3 can have at least about 50%, 55%, 60%, 65%, 70%, 75%, 80%, 85%, 90%, 95%, 98%, 99% or 100% sequence identity with SEQ ID NO: 5. SEQ ID NO: 5 is the Arabidopsis CHK3 protein.

Preferably, the nucleotide sequence encoding the CHK4 preferably has at least about 50%, 55%, 60%, 65%, 70%, 75%, 80%, 85%, 90%, 95%, 98%, 99% or 100% sequence identity with SEQ ID NO: 6. SEQ ID NO: 6 is the Arabidopsis CHK4 protein.

In one embodiment, the CHK2 amino acid sequence is or is derived from AT5G35750.1 (AHK2), a homolog thereof, or a sequence having at least about 50%, 55%, 60%, 65%, 70%, 75%, 80%, 85%, 90%, 95%, 98%, 99% or 100% sequence identity with AHK2 or its homolog. A preferred homolog is Solanum lycopersicum CHK2 (SlyCHK2) having a sequence of SEQ ID NO: 31, and/or a sequence having at least about 50%, 55%, 60%, 65%, 70%, 75%, 80%, 85%, 90%, 95%, 98%, 99% or 100% sequence identity with SlyCHK2.

Examples of homologs include Glycine max (Soybean) (K7KBR1 or K7M476), Oryza sativa (Rice) (Q0IY65, Q9AUQ0 or Q8S6P5), Populus trichocarpa (Black Cottonwood) (B9IAR0), Solanum lycopersicum (Tomato) (K4CEY3) and Vitis vinifera (Grape) (F6HHM7).

In one embodiment, the CHK3 amino acid sequence is or is derived from AT1G27320 (AHK3), a homolog thereof, or a sequence having at least about 50%, 55%, 60%, 65%, 70%, 75%, 80%, 85%, 90%, 95%, 98%, 99% or 100% sequence identity with AHK3 or its homolog.

Examples of homologs include Brachypodium distachyon (Purple false brome) (I1HUP8), Glycine max (Soybean) (I1K3M7 or I1KS30), Oryza sativa (Rice) (Q5JJP1), Populus trichocarpa (Black Cottonwood) (B9GML7 or B9GZP2), Solanum lycopersicum (Tomato) (K4BYS7) and Vitis vinifera (Grape) (D7TAZ7).

In one embodiment, the CHK4 amino acid sequence is or is derived from AT2G01830 (AHK4), a homolog thereof, or a sequence having at least about 50%, 55%, 60%, 65%, 70%, 75%, 80%, 85%, 90%, 95%, 98%, 99% or 100% sequence identity with AHK4 or its homolog.

Examples of homologs include Brachypodium distachyon (Purple false brome) (I1GNZ3), Glycine max (Soybean) (K7K767, K7KRH0, K7L210, K7L2C5 or I1KQE9), Physcomitrella patens (Moss) (A9RME1, A9SJM1, A9T3T9, A9S5U9, A9TAF3, A9TKN3, A9S2L4, A9TCH3, A9TVMO, A9SEU1 or A9RME0), Populus trichocarpa (Black Cottonwood) (B9HVS3 or B9HJJ3), Solanum lycopersicum (Tomato) (K4BNW7), Sorghum bicolor (Sorghum) (C5WN04) and Vitis vinifera (Grape) (F6HFB2)

In an embodiment of the invention, the histidine kinase having increased or introduced expression is a functional histidine kinase. A functional histidine kinase is preferably fulfilling the same or similar function in a plant cell as the function of a histidine kinase having amino acid sequence SEQ ID NO: 4, SEQ ID NO: 5 or SEQ ID NO: 6 in Arabidopsis thaliana. A CHK2 as defined herein is preferably fulfilling the same or similar function in a plant cell as the function of a protein having amino acid sequence of SEQ ID NO: 4 in Arabidopsis thaliana. A CHK3 as defined herein is preferably fulfilling the same or similar function in a plant cell as the function of a protein having amino acid sequence of SEQ ID NO: 5 in Arabidopsis thaliana. A CHK4 as defined herein is preferably fulfilling the same or similar function in a plant cell as the function of a protein having amino acid sequence of SEQ ID NO: 6 in Arabidopsis thaliana. In context of the invention, a functional histidine kinase, or CHASE-domain containing histidine kinase, is preferably capable of binding cytokinin and initiates a signal transduction that results in cytokinin-regulated transcription. Alternatively or in addition, the functional histidine kinase can also be defined as a histidine kinase that is capable of improving the cytokinin-induced regeneration capacity of a plant cell upon increased or introduced expression.

The method can comprise, for example, a step of genetically engineering the plant, plant protoplast or plant cell to overexpress or express de novo a histidine kinase protein. In one embodiment, the expression of the histidine kinase is transiently increased or introduced into the plant cell. It is well-known in the art how to transiently increase or introduce the expression of a protein and the invention is not limited to any specific method.

As a non-limiting example, the method can comprise transforming a plant cell with a vector or expression construct comprising a recombinant nucleic acid encoding the histidine kinase as described herein, preferably as an expression construct of the ninth aspect as defined herein. Such vector or expression construct preferably does not integrate into the plant genome. As a result, the histidine kinase protein transcribed from the expression construct will be temporarily expressed. Introduction of a nucleic acid encoding the histidine kinase can be performed using any suitable means known in the art, for example such as described in WO2009/082190. In some embodiments, the method comprises contacting the plant protoplast or plant cell with an Agrobacterium strain comprising the vector to introduce the recombinant nucleic acid into the plant protoplast or plant cell.

Alternatively or in addition, the plant cells can be transformed with a histidine kinase protein as defined herein. The introduction of the histidine kinase protein can be performed by any suitable means known to the skilled person.

In one embodiment, the genome of the plant cell can be modified to transiently express or overexpress the histidine kinase protein as defined herein. As a non-limiting example, an expression cassette can be introduced into the genome of a plant cell, wherein the expression cassette at least comprises an inducible promoter and a sequence encoding the histidine kinase. The histidine kinase can for example be expressed upon the presence of an inducer. The inducer can bind to a transactivator, e.g. an introduced transactivator, which transactivator initiates or augments the expression of the histidine kinase, for example by binding to the inducible promoter that is operably linked to the sequence encoding the histidine kinase.

In an embodiment, the expression of the histidine kinase is continuously increased or introduced in the plant cell.

Continuous overexpression or continuous de novo expression of the histidine kinase protein can be achieved by, for example, inserting at least one additional copy of an endogenous gene encoding the histidine kinase protein into the genome of a plant, plant protoplast or plant cell. Further ways are modulating promoter and/or further regulating sequences that are operably linked to an endogenous histidine kinase-encoding sequence, resulting in enhanced or introduced expression. These regulating sequences can include genomic as well as epigenomic regulators.

In an embodiment, the genome of the plant is modified to overexpress or de novo express a histidine kinase as defined herein by modifying a genomic promoter fragment having at least about 50%, 55%, 60%, 65%, 70%, 75%, 80%, 85%, 90%, 95%, 98% or 100% sequence identity with at least one of SEQ ID NO: 10, SEQ ID NO: 11 and SEQ ID NO: 12.

In one embodiment, the genome of the plant cell is modified to overexpress or de novo express a histidine kinase as defined herein. As a non-limiting example, regulatory sequences surrounding a gene encoding an endogenous histidine kinase as defined herein can be modified to increase or induce the expression of the histidine kinase. These regulatory sequences can be located less than about 10 kb, 8 kb, 5 kb, 3 kb, 2 kb, 1 kb, 800 bp, 600 bp, 400 bp, 200 bp, 100 bp or less than about 50 bp upstream from the start codon encoding the histidine kinase. Alternatively, these regulatory sequences can be located less than about 10 kb, 8 kb, 5 kb, 3 kb, 2 kb, 1 kb, 800 bp, 600 bp, 400 bp, 200 bp, 100 bp or less than about 50 bp downstream from the start codon encoding the histidine kinase.

In an embodiment, the regulatory sequences modified to increase or induce the expression of an endogenous histidine kinase as defined herein can be located less than about 10 kb, 8 kb, 5 kb, 3 kb, 2 kb, 1 kb, 800 bp, 600 bp, 400 bp, 200 bp, 100 bp or less than about 50 bp upstream of a sequence having at least about 50%, 60%, 70%, 80%, 90%, 95%, 98% or 100% sequence identity with SEQ ID NO: 7.

In an embodiment, the regulatory sequences modified to increase or induce the expression of an endogenous histidine kinase as defined herein can be located less than about 10 kb, 8 kb, 5 kb, 3 kb, 2 kb, 1 kb, 800 bp, 600 bp, 400 bp, 200 bp, 100 bp or less than about 50 bp upstream of a sequence having a sequence with at least about 50%, 60%, 70%, 80%, 90%, 95%, 98% or 100% sequence identity with SEQ ID NO: 8.

In an embodiment, the regulatory sequences modified to increase or induce the expression of an endogenous histidine kinase as defined herein can be located less than about 10 kb, 8 kb, 5 kb, 3 kb, 2 kb, 1 kb, 800 bp, 600 bp, 400 bp, 200 bp, 100 bp or less than about 50 bp upstream of a sequence having a sequence with at least about 50%, 60%, 70%, 80%, 90%, 95%, 98% or 100% sequence identity with SEQ ID NO: 9.

In an embodiment, the plant genome can be modified at specific position using for example KeyBase®, targeted nucleotide exchange (TNE), oligo-directed mutagenesis (ODM) or Oligonucleotide Directed Targeted Mutagenesis (ODTM) to increase or introduce the expression of a histidine kinase as defined herein.

In some embodiment, methods for editing the genome of the plant cell to introduce or increase the expression of an endogenous histidine kinase as defined herein includes, but is not limited to, the use of specific nucleases such as the CRISPR system, ZFNs or TALENs. Non-limiting examples of specific nucleases of the CRISPR system include Cas9, Cpf1 and CasX.

Instead or in addition to introducing or increasing the expression of an endogenous histidine kinase, an exogenous histidine kinase protein can be stably expressed in a plant cell. For example, a nucleic acid encoding a histidine kinase, e.g. an exogenous histidine kinase, can be stably inserted into the genome of a plant cell. The expression of such histidine kinase can be regulated by sequences operably linked to the introduced sequence. These regulating sequences can be endogenous to the cell, or may be introduced e.g. together with the introduction of the nucleic acid encoding the histidine kinase. These regulating sequences can include genomic as well as epigenomic regulators.

The method of the invention may further comprise a step of testing overexpression or de novo expression of the histidine kinase of the method of the invention. In other words, the invention also provides for a method for improving a cytokinin-induced regeneration capacity of a plant cell comprising the steps of:
a) increasing or introducing the expression of a histidine kinase as defined herein in the plant cell; and,
b) detecting the expression level of said histidine kinase in the plant cell.

Methods for testing overexpression or de novo expression of the histidine kinase protein include, but are not limited to, PCR analysis, sequencing of genomic DNA, sequencing of mRNA transcript, analyzing mRNA transcript levels (Northern-blot analysis), analyzing copy number (Southern blot analysis), etc. Preferably, the method of the invention results in at least 2%, 5%, 10%, 20%, 30%, 40%, 50%, 60%, 70%, 80%, 90%, or 100% increase of expression of the histidine kinase as compared to the control as defined herein.

In an embodiment, the plant cell is obtained or obtainable from a plant that is known to have an inefficient regeneration capacity. An insufficient regeneration capacity is herein understood as the regeneration capacity is less than about 25%, 20%, 15%, 10%, 8%, 5%, 4%, 3%, 2%, or 1%, preferably when the plant cell is kept under defined conditions, preferably optimal conditions, that allow for regeneration. Examples of plants having an inefficient regeneration capacity include, but are not limited to sweet pepper, cucumber and melon.

The plant cell for use in the invention may be obtained or obtainable from a plant that is incapable to regenerate under conditions that should allow for regeneration.

In one embodiment, the plant cell is obtained or obtainable from a plant selected from the group consisting of barley, cabbage, canola, cassava, cauliflower, chicory, cotton, cucumber, eggplant, grape, hot pepper, lettuce, maize, melon, oilseed rape, potato, pumpkin, rice, rye, sorghum, squash, sugar cane, sugar beet, sunflower, sweet pepper, tomato, water melon, wheat, zucchini, soybean, chrysanthemum and Arabidopsis.

In one embodiment, the plant cell is obtained or obtainable from a plant selected from the group consisting of sweet pepper, cucumber, melon, soybean, chrysanthemum and Arabidopsis. In an embodiment, the plant cell is obtained or obtainable from a plant selected from the group consisting of sweet pepper, cucumber and melon.

In one embodiment, the plant cell is obtained or obtainable from tomato or pepper, preferably the plant cell is obtained or obtainable from Solanum lycopersicum or Capsicum annuum.

In a second aspect, the invention pertains to a method for regenerating a plant comprising the steps of i) incubating a plant cell obtained or obtainable by the method of the first aspect of the invention in a medium comprising a cytokinin as defined herein; and ii) allowing the plant cell to regenerate into a plant.

The invention therefore also pertains to a method for regenerating a plant comprising the steps of:
a) increasing or introducing the expression of a histidine kinase as defined herein in the plant cell;
b) optionally detecting the expression level of said histidine kinase in the plant cell and optionally selecting a plant cell having an increased or introduced expression of the histidine kinase;
c) incubating the plant cell, optionally the plant cell selected in step b), in a medium comprising a cytokinin as defined herein; and
d) allowing the plant cell to regenerate into a plant.

In one embodiment, the plant cell is a plant cell as specified in the first aspect of the invention. The plant cell can be obtained or obtainable from a plant selected from the group consisting of barley, cabbage, canola, cassava, cauliflower, chicory, cotton, cucumber, eggplant, grape, hot pepper, lettuce, maize, melon, oilseed rape, potato, pumpkin, rice, rye, sorghum, squash, sugar cane, sugar beet, sunflower, sweet pepper, tomato, water melon, wheat, zucchini, soybean, chrysanthemum and Arabidopsis.

The method of the invention relates to improving a cytokinin-induced regeneration capacity of a plant cell, wherein the method comprises a step of increasing or introducing the expression of a histidine kinase protein. Cytokinins (CK) are a class of plant growth substances (phytohormones) that promote cell division, or cytokinesis, in plant roots and shoots. Cytokinins can travel up the xylem and promote lateral growth. They are involved primarily in cell growth and differentiation, but can also affect apical dominance, axillary bud growth, and leaf senescence.

The cytokinin for use in the invention can be an adenine-type cytokinin or a phenylurea-type cytokinin. Similarly, the cytokinin can be a naturally produced phytohormone or can be a synthesized compound. The adenine-type cytokinin can be a phytohormone that is synthesized in at least one of roots, seeds and fruits. In addition, cambium and other actively dividing tissues can also synthesize cytokinins

A non-limiting example of a naturally occurring adenine-type cytokinin is Zeatin as well as its metabolic precursor 2iP. Non-limiting examples of synthetic adenine-type cytokinins are kinetin and 6-benzylaminopurine (BAP). Substituted urea compounds, such as thidiazuron and CPPU do not occur in plants but can act as cytokinins in tissue culture.

The adenine-type cytokinin can be selected from the group consisting of kinetin, zeatin, trans-zeatin, cis-zeatin, dihydrozeatin, 6-benzylaminopurine and 2iP, and combinations thereof. The phenylurea-type cytokinin can be diphenylurea or thidiazuron.

In one embodiment, the plant cell as defined herein is incubated in a medium comprising a cytokinin as defined herein under conditions that allow for the regeneration of the plant cell. The plant cell can be incubated in the medium comprising a cytokinin for at least about 1, 2, 3, 4, 5, 6, 7, 8, 9 or about 10 days, under conditions that allow for regeneration. The plant cell can be incubated in the medium comprising a cytokinin for at least about 1, 2, 3, 4, 5, 6, 7, 8, 9, 10, 11 or about 12 weeks, under conditions that allow for regeneration.

In one embodiment, the plant cell as defined herein is incubated in a medium comprising a cytokinin for at least 5, 6, 7, 8, 9 weeks.

In one embodiment, the regenerated plant cell can be further developed into a cotyledonary embryo. The terms “cotyledonary embryo” and “cotyledonary stage embryo” can be used interchangeably herein. The cotyledonary embryo is preferably a cotyledonary stage somatic embryo. In an embodiment, all cells of the cotyledonary embryo express or overexpress the histidine kinase as defined herein. The mutagenized cotyledonary embryo produced by the method of the invention can be further developed into a plantlet. The plantlet can further be developed into a plant.

In an embodiment of the invention, the method therefore comprises a further step of regenerating a plantlet and/or a plant from the plant cell.

In one embodiment, the plant cell as defined herein is incubated in a medium comprising a cytokinin. The cytokinin in the medium can be only one type of cytokinin. Alternatively, the plant cell can also be incubated in a mixture of cytokinins. For example, the plant cell can be incubated in a medium comprising at least 1, 2, 3, 4 or 5 different types of cytokinins.

A mixture of at least two cytokinins is for example selected from the group consisting of kinetin and zeatin, kinetin and trans-zeatin, kinetin and cis-zeatin, kinetin and dihydrozeatin, kinetin and 6-benzylaminopurine, kinetin and 2iP, zeatin and trans-zeatin, zeatin and cis-zeatin, zeatin and dihydrozeatin, zeatin and 6-benzylaminopurine, zeatin and 2iP, trans-zeatin, and cis-zeatin, trans-zeatin and dihydrozeatin, trans-zeatin and 6-benzylaminopurine, trans-zeatin and 2iP, cis-zeatin and dihydrozeatin, cis-zeatin and 6-benzylaminopurine, cis-zeatin and 2iP, dihydrozeatin and 6-benzylaminopurine, dihydrozeatin and 2iP, and 6-benzylaminopurine and 2iP. The skilled person understands that (an) additional type(s) of cytokinin(s) can be added to the mixture of the at least two cytokinins. It is known in the art that the type of added cytokinin is dependent on the type of plant cell and the skilled person can straightforwardly select the suitable cytokinin(s).

In one embodiment, the medium can contain additional compounds that promote the regeneration of the plant cell. Such additional compounds can for example be one or more growth regulators, e.g. one or more plant hormones. Plant hormones (also known as phytohormones or ‘plant growth substances’) are chemicals that can regulate plant growth. A plant hormone can affect at least one of plant shape, seed growth, flowering time, the sex of flowers, senescence of leaves, and senescence of fruits. Alternatively or in addition, plant hormone can affect at least one of upward growth of tissues, downward growth of tissues, leaf formation, stem growth, fruit development, fruit ripening, plant longevity, and plant death.

The further plant hormone for use in the method of the invention can be at least one of an auxin, abscisic acid, ethylene and a gibberellin. Alternatively or in addition, the further plant hormone can be at least one of a brassinosteroid, salicylic acid, a jasmonate, a plant peptide hormone, a polyamine, nitric oxide, a strigolactones, a Karrikin and triacontanol.

The at least one further plant hormone can be an auxin. Auxins are a class of plant hormones that can have morphogen-like characteristics. The auxin can be an endogenously synthesized auxin. The endogenously synthesized auxin can be selected from the group consisting of indole-3-acetic acid (IAA), 4-chloroindole-3-acetic acid, phenylacetic acid, indole-3-butyric acid and indole-3-propionic acid.

The auxin can be a synthetic auxin, e.g. an auxin analog. The synthetic auxin can be at least one of 1-naphthaleneacetic acid, 2,4-dichlorophenoxyacetic acid (2,4-D), α-Naphthalene acetic acid (α-NAA), 2-Methoxy-3,6-dichlorobenzoic acid (dicamba), 4-Amino-3,5,6-trichloropicolinic acid (tordon or picloram), 1-naphthaleneacetic acid (NAA), indole-3-butyric acid (IBA) and 2,4,5-trichlorophenoxyacetic acid (2,4,5-T). The auxin can be 1-naphthaleneacetic acid (NAA).

In addition or alternatively, the further plant hormone can be at least a gibberellin. The gibberellin can be a 19-carbon gibberellin or a 20-carbon gibberellin. The gibberellin can be a dihydroxylated gibberellin. The gibberellin can be at least one of GA1, GA3, GA4 and GA7.

The concentration of the at least one further plant hormone (e.g. auxin) in the medium can be the same or similar to the concentration cytokinin (e.g. a ratio of about 1:1). Alternatively, the concentration of the at least one further plant hormone can be lower than the concentration cytokinin in the medium. The ratio of the at least one further plant hormone to cytokinin can be about 0.9:1.0; 0.8:1.0; 0.7:1.0; 0.6:1.0; 0.5:1.0; 0.4:1.0; 0.3:1.0; 0.2:1.0; 0.1:1.0; 0.01:1.0 or even about 0.001:1.0.

Alternatively, the concentration of the at least one further plant hormone can be higher than the concentration cytokinin in the medium. The ratio of the at least one further plant hormone to cytokinin can be about 1:0.9; 1:0.8; 1:0.7; 1:0.6; 1:0.5; 1:0.4; 1:0.3; 1:0.2; 1:0.1; 1:0.01 or even about 1:0.001.

The concentration cytokinin in the medium can be at least about 50 ng/ml, 75 ng/ml, 100 ng/ml, 125 ng/ml, 150 ng/ml, 175 ng/ml, 200 ng/ml, 225 ng/ml, 250 ng/ml, 275 ng/ml, 300 ng/ml, 325 ng/ml ng/ml, 350 ng/ml, 375 ng/ml, 400 ng/ml, 425 ng/ml, 450 ng/ml, 475 ng/ml, 500 ng/ml, 525 ng/ml, 550 ng/ml, 575 ng/ml, 600 ng/ml, 625 ng/ml, 650 ng/ml, 675 ng/ml, 700 ng/ml, 750 ng/ml, 800 ng/ml, 850 ng/ml, 900 ng/ml, 950 ng/ml, 1000 ng/ml, 1100 ng/ml, 1200 ng/ml, 1300 ng/ml, 1400 ng/ml, 1500 ng/ml, 1750 ng/ml, 2000 ng/ml, 2225 ng/ml, 2500 ng/ml, 2750 ng/ml, 3000 ng/ml, 3250 ng/ml, 3500 ng/ml, 3750 ng/ml, 4000 ng/ml, 4250 ng/ml, 4500 ng/ml, 4750 ng/ml or at least about 5000 ng/ml.

Alternatively or in addition, the concentration cytokinin in the medium can at most about 50 ng/ml, 75 ng/ml, 100 ng/ml, 125 ng/ml, 150 ng/ml, 175 ng/ml, 200 ng/ml, 225 ng/ml, 250 ng/ml, 275 ng/ml, 300 ng/ml, 325 ng/ml ng/ml, 350 ng/ml, 375 ng/ml, 400 ng/ml, 425 ng/ml, 450 ng/ml, 475 ng/ml, 500 ng/ml, 525 ng/ml, 550 ng/ml, 575 ng/ml, 600 ng/ml, 625 ng/ml, 650 ng/ml, 675 ng/ml, 700 ng/ml, 750 ng/ml, 800 ng/ml, 850 ng/ml, 900 ng/ml, 950 ng/ml, 1000 ng/ml, 1100 ng/ml, 1200 ng/ml, 1300 ng/ml, 1400 ng/ml, 1500 ng/ml, 1750 ng/ml, 2000 ng/ml, 2225 ng/ml, 2500 ng/ml, 2750 ng/ml, 3000 ng/ml, 3250 ng/ml, 3500 ng/ml, 3750 ng/ml, 4000 ng/ml, 4250 ng/ml, 4500 ng/ml, 4750 ng/ml or about 5000 ng/ml.

In an embodiment, the concentration cytokinin in the medium is in the range of about 50-5000 ng/ml, 75-4000 ng/ml, 100-3000 ng/ml, 125-2000 ng/ml, 130-1500 ng/ml, 140-1250 ng/ml, 150-1000 ng/ml, 175-800 ng/ml, 200-600 ng/ml or about 250-500 ng/ml.

The concentration cytokinin in the medium is preferably a concentration that is optimal to allow regeneration of a plant cell. The skilled person knows how to establish such optimal concentrations, e.g. through routine experimentation or these concentrations have been described previously in the art. A preferred optimal concentration is about 1000 ng/ml.

The medium for incubating the plant cell as defined herein can be a liquid medium or a solid medium. The medium is preferably sterile.

In the method and use of the invention as defined herein in the different aspects, the plant cell can be part of a multicellular tissue. A plant multicellular tissue can comprise differentiated cells. Alternatively or in addition, a multicellular tissue can comprise undifferentiated cells. In an embodiment, all cells of the multicellular tissue have an increased or introduced expression of a histidine kinase as defined herein. Alternatively, about 5%, 10%, 15%, 20%, 25%, 30%, 35%, 40%, 45%, 50%, 55%, 60%, 65%, 70%, 75%, 80%, 85%, 90%, 95%, 97%, 98% or about 99% of the cells of the multicellular tissue have an increased or de novo expression of a histidine kinase as defined herein.

The multicellular tissue can be a callus tissue, a plant organ or an explant.

The plant cell having induced or introduced expression of a histidine kinase as defined herein can be part of a callus. A callus is a group of undifferentiated cells, preferably derived from adult cells. Callus cells can be capable of undergoing embryogenesis and formation of an entirely new plant. A plant callus is considered a growing mass of unorganized plant parenchyma cells. Callus can be produced from a single differentiated cell, and callus cells can be totipotent, being able to regenerate the whole plant body. The plant callus can be derived from a somatic tissue or tissues, e.g. a tissue that is available for explant culture. The cells that give rise to callus and somatic embryos preferably undergo rapid division and/or are partially undifferentiated such as meristematic tissue. The callus cell used in the method of the invention can be friable or compact. In addition or alternatively the callus cell can be rooty, shooty, or embryogenic callus (Ikeuchi M, Plant Cell. 2013 September; 25 (9): 3159-3173).

In an embodiment, the plant cell having an increased or introduced expression of a histidine kinase as defined herein can be part of a plant organ. The plant organ can be a vegetative organ or a reproductive organ. A vegetative organ can be derived from the shoot system or root system. The organ can be at least one of roots, stems and leaves. A reproductive plant organ can be selected from the group consisting of flower, seed, fruit, cone, sori, strobili and gametophores.

In an embodiment, the plant cell having an increased or introduced expression of a histidine kinase as defined herein can be part of an explant. An explant can be defined herein as a sample obtained from a part of a plant. The plant sample can be placed on a solid culture medium or liquid medium. Explants can be taken from many different parts of a plant, including portions of shoots, leaves, stems, flowers, roots, single undifferentiated cells and from mature cells. The cells preferably contain living cytoplasm and nuclei and are able to de-differentiate and resume cell division. An explant can be, or can be obtainable or obtained from, a meristematic end of a plant, such as e.g. the stem tip, axillary bud tip or root tip. In one embodiment, the explant is selected from the group consisting of a hypocotyl explant, a stem explant, a cotyledon explant, a root explant, a leaf explant, a flower explant and a meristematic tissue. In one embodiment, the explant is a hypocotyl explant.

The plant regenerated by the method of the invention may subsequently be crossed to remove the increased or introduced expression of a histidine kinase as defined herein. Hence the method may comprise a step of crossing the regenerated plant to remove the previously increased or introduced expression of a histidine kinase. The plant may be crossed with a plant of a different species or of the same species and progeny that no longer has an increased or introduced expression of a histidine kinase may be selected.

In a third aspect, the invention pertains to a plant or plant part obtainable or obtained by the method of the invention as defined herein. The plant or plant part can be obtainable or obtained by the process of organogenesis or somatic embryogenesis. Plant cells obtained from the plant or plant part have an increased or introduced expression of a histidine kinase as defined herein. The plant obtainable or obtained by the method of the invention is preferably selected from the group consisting of barley, cabbage, canola, cassava, cauliflower, chicory, cotton, cucumber, eggplant, grape, hot pepper, lettuce, maize, melon, oilseed rape, potato, pumpkin, rice, rye, sorghum, squash, sugar cane, sugar beet, sunflower, sweet pepper, tomato, water melon, wheat, zucchini, soybean, chrysanthemum and Arabidopsis. The plant, plant part or plant cell can have an increased or induced expression of the histidine kinase as defined herein in all cells. Alternatively about 5%, 10%, 15%, 20%, 25%, 30%, 35%, 40%, 45%, 50%, 55%, 60%, 65%, 70%, 75%, 80%, 85%, 90%, 95%, 97%, 98% or about 99% of the plant cells have an increased or introduced expression of the histidine kinase as defined herein. In one embodiment, the plant part is a seed, a fruit or a non-propagating material.

In an embodiment, the plant or the plant cell of the invention comprises a genetic modification, wherein the modification results in an increased or introduced expression of a histidine kinase, as compared to a control plant. Preferred genetic modifications are indicated herein above. The plant or plant cell of the invention preferably comprises a nucleic acid of the eight aspect as defined herein and/or an expression construct of the ninth aspect as defined herein.

In a fourth aspect, the invention concerns a product derived from the plant or plant part obtainable or obtained by the method of the invention, e.g. fruits, leaves, plant organs, plant fats, plant oils, plant starch, and plant protein fractions, either crushed, milled or still intact, mixed with other materials, dried, frozen, and so on. These products may be non-propagating. Preferably, said plant product comprises a genetic modification, wherein the modification results in an increased or introduced expression of a histidine kinase, as compared to a control plant. Preferred genetic modifications are indicated herein above. Said plant product may comprise a nucleic acid of the eight aspect as defined herein and/or an expression construct of the ninth aspect as defined herein. Preferably, these products comprise at least fractions of said genetic modification, nucleic acid and/or construct, which allows to assess that the plant product is derived from a plant obtained by the method of the first and/or second aspect of the invention as defined herein.

In a fifth aspect, the invention concerns progeny of the plant cell, plantlet or plant obtainable or obtained by the method of the invention. Hence, the plant cells of the progeny have an increased or induced expression of a histidine kinase as defined herein and have an improved cytokinin-induced regeneration capacity. Said progeny may comprise the genetic modification, nucleic acid and/or an expression construct of the plant or plant part of the third aspect of the invention as defined herein.

In a sixth aspect, the invention pertains to the use of a CHK2, CHK3 and/or CHK4 histidine kinase, as defined herein for improving a cytokinin-induced regeneration capacity of a plant. In one embodiment, the invention concerns the use of a CHK2 and/or CHK4 histidine kinase as defined herein for improving a cytokinin-induced regeneration capacity of a plant. In a further embodiment, the invention concerns the use of at least a CHK4 histidine kinase as defined herein for improving a cytokinin-induced regeneration capacity of a plant. The plant is preferably a plant as defined herein above.

In a seventh aspect, the invention pertains to the use of a CHK2, CHK3 and/or CHK4 histidine kinase as defined herein above in a method for improving a cytokinin-induced regeneration capacity of a plant. Preferably, the CHK2, CHK3 and/or CHK4 histidine kinase is used in a method as defined in the first and/or second aspect of the invention, for improving a cytokinin-induced regeneration capacity of a plant.

In an eighth aspect, the invention concerns a nucleic acid encoding a histidine kinase as defined herein. In a preferred embodiment, the nucleic acid encodes for at least one of CHK2, CHK3 and CHK4 as defined herein. Preferably the nucleic acid encodes for least one of CHK2 and CHK4 as defined herein. Preferably, the nucleic acid encodes for CHK4 as defined herein.

In one embodiment, the sequence of the nucleic acid can have at least about 40%, 45%, 50%, 55%, 60%, 65%, 70%, 75%, 80%, 85%, 90%, 95%, 98% or about 100% sequence identity with at least one of SEQ ID NO: 1, SEQ ID NO: 2 and SEQ ID NO: 3.

Alternatively, the sequence of the nucleic acid can have at least about 40%, 45%, 50%, 55%, 60%, 65%, 70%, 75%, 80%, 85%, 90%, 95%, 98% or about 100% sequence identity with at least one of SEQ ID NO: 7, SEQ ID NO: 8 and SEQ ID NO: 9.

The nucleic acid can be isolated from its natural environment. In addition or alternatively, at least 1, 2, 3 or 4 nucleotides can flank the nucleic acid as defined herein, wherein said nucleotides do not flank the nucleic acid in a natural environment, i.e. resulting in a non-naturally occurring nucleic acid.

In a ninth aspect, the invention pertains to an expression construct for the expression of a histidine kinase as defined herein. In an embodiment, the expression construct comprises the sequence of a nucleic acid as defined herein above in the eighth aspect. The expression construct can comprise the sequence of at least 1, 2, 3, 4 or 5 nucleic acids as defined herein above in the eighth aspect.

The expression construct can comprise the sequence of at least two or more copies of the same nucleic acid and/or can comprise the sequence of at least two different nucleic acids as defined herein.

As a non-limiting example, the expression construct can comprise at least the sequence of a nucleic acid having at least about 40%, 45%, 50%, 55%, 60%, 65%, 70%, 75%, 80%, 85%, 90%, 95%, 98% or about 100% sequence identity with SEQ ID NO: 1 as well as the sequence of a nucleic acid having at least about 40%, 45%, 50%, 55%, 60%, 65%, 70%, 75%, 80%, 85%, 90%, 95%, 98% or about 100% sequence identity with SEQ ID NO: 3,

As an another non-limiting example, the expression construct can comprise at least the sequence of a nucleic acid having at least about 40%, 45%, 50%, 55%, 60%, 65%, 70%, 75%, 80%, 85%, 90%, 95%, 98% or about 100% sequence identity with SEQ ID NO: 7 as well as the sequence of a nucleic acid having at least about 40%, 45%, 50%, 55%, 60%, 65%, 70%, 75%, 80%, 85%, 90%, 95%, 98% or about 100% sequence identity with SEQ ID NO: 9,

The expression construct can further comprise one or more regulatory elements that are operably linked to the nucleic acid sequence or nucleic acid sequences as defined herein. A preferred regulatory element is a promoter. The promoter for expression in a plant cells is herein understood as a promoter that is active in plants or plant cells, i.e. the promoter has the general capability to drive transcription within a plant or plant cell.

The promoter can be a constitutive promoter, an inducible promoter or a tissue specific promoter. It is understood herein that a “constitutive” promoter is a promoter that is active in most tissues under most physiological and developmental conditions. An “inducible” promoter is a promoter that is physiologically (e.g. by external application of certain compounds) or developmentally regulated. A “tissue specific” promoter is only active in specific types of tissues or cells.

The promoter can be a caulimovirus promoter, such as a cauliflower mosaic virus (CaMV) promoter, a nopaline synthase promoter or an octopine synthase promoter.

The promoter can have at least about 40%, 45%, 50%, 55%, 60%, 65%, 70%, 75%, 80%, 85%, 90%, 95%, 98% or about 100% sequence identity with at least one of SEQ ID NO: 10, SEQ ID NO: 11, SEQ ID NO: 12 and SEQ ID NO: 13. Preferably, the promoter can have at least about 40%, 45%, 50%, 55%, 60%, 65%, 70%, 75%, 80%, 85%, 90%, 95%, 98% or about 100% sequence identity with SEQ ID NO: 12.

The present invention has been described above with reference to a number of exemplary embodiments. Modifications and alternative implementations of some parts or elements are possible, and are included in the scope of protection as defined in the appended claims.

### EXAMPLES

### Example 1: Cloning of the Arabidopsis CHK2 Gene

The coding sequence of the Arabidopsis CHK2 gene (AT5G35750) was amplified by PCR using CHK2 specific forward primer 14_04109 and reverse primer 14_04110 from Arabidopsis thaliana Col-0 genomic DNA as template. A second PCR amplification was performed with primers 14_04116 (forward) and 14_04117 (reverse) to incorporate Gateway cloning attB sites at the ends of the fragments. The amplification products were purified from gel and cloned into pDONR221 donor vector (Invitrogen™) via standard Gateway BP reaction cloning (thermofisher).

The 2.0 kb promoter sequence upstream of the AtCHK2 gene (AT5G35750) was synthesized by gene synthesis (ThermoFisher Scientific). The synthesized fragment was PCR-amplified using forward primer 15_00083 and reverse primer 15_00084, and cloned into entry vector pENTR™ 5′-TOPO using the pENTR™ 5′-TOPOR TA Cloning® Kit (Invitrogen™).

### Example 2: Cloning of the Arabidopsis CHK4 Gene

The coding sequence of the Arabidopsis CHK4 gene (AT2G01830) was amplified by PCR using CHK4 specific forward primer 14_04106 and reverse primer 14_04107 from Arabidopsis thaliana Col-0 genomic DNA as template. A second amplification was performed with primers 14_04114 (forward) and 14_04115 (reverse) to incorporate Gateway cloning attB sites at the ends of the fragments. The amplification products were purified from gel and cloned into pDONR221 donor vector (Invitrogen™) via standard Gateway BP reaction cloning thermofisher).

The 2.0 kb promoter sequence upstream of the AtCHK4 gene (AT2G01830) was synthesized by gene synthesis (ThermoFisher Scientific). The synthesized fragment was PCR-amplified using forward primer 15_00087 and reverse primer 15_00088, and cloned into entry vector pENTR™ 5′-TOPO using the pENTR™ 5′-TOPOR TA Cloning® Kit (Invitrogen™).

### Example 3: Construction of Expression Constructs pKG9785 and pKG9791

An expression construct for the Arabidopsis CHK2 gene under control of its native 2 kb promoter was obtained by combining the CHK2 promoter fragment and the CHK2 coding sequence fragment from the entry vectors described in Example 1 into multisite Gateway destination vector pK7m24gw,3 through a single step LR cloning reaction (ThermoFisher).

An expression construct for the Arabidopsis CHK4 gene under control of its native 2 kb promoter was obtained by combining the CHK4 promoter fragment and the CHK4 coding sequence fragment from the entry vectors described in Example 2 into multisite Gateway destination vector pK7m24gw,3 through a single step LR cloning reaction (ThermoFisher).

The resulting plasmid constructs containing either CHK coding sequence under control of its native promoter were checked using restriction enzyme digestion with the combination NcoI and SacI, or the combination XbaI and HindIII, prior to transformation to Agrobacterium tumefaciens strain GV3101 via electroporation. The final constructs were named pKG9785 for the CHK2 expression cassette and pK9791 for the CHK4 expression cassette, respectively. Individual Agrobacterium colonies were selected and used in plant transformation to obtain transgenic lines.

### Example 4: Transformation of Arabidopsis

Transgenic plants with stable integration of either CHK construct (pKG9785 or pKG9791) were obtained by transforming Arabidopsis thaliana Col-0 plants using the floral dip method according to the protocol of Clough and Bent (Plant J. 16 (6): 735-743, 1998). Positive transformants were obtained by sterilizing T1 progeny seed using vapor phase (Clough and Bent, 1998, Plant J. 16 (6): 735-743), germinating the seeds in vitro on 0.5 MS10 culture medium (which is half strength MS medium according to Murashige and Skoog, 1962, Physiol. Plant. 15:473-497 containing 100 g·L⁻¹ sucrose) supplemented with 50 mg·L⁻¹ kanamycin, and selecting kanamycin-resistant seedlings. Multiple independent kanamycin-resistant seedlings for each construct were subsequently transferred to soil and selfed to obtain T2 seeds. Seeds were sterilized using vapor phase and sown on 0.5 MS10 plates supplemented with 50 mg·L⁻¹ kanamycin. After two weeks, T3 lines of which the seedlings did not segregate for kanamycin resistance or sensitivity were considered homozygous and used in the regeneration assays. Expression levels of the transgenes in selected transgenic lines was checked by qRT-PCR using RNA extracted from whole in vitro seedlings and compared to the expression levels of the endogenous CHK genes in non-transformed Col-0 control plants. The primers used for qRT-PCR were 17_03894 (forward primer) and 17_03895 (reverse primer) for AtCHK4, and 17_03890 (forward) and 17_03891 (reverse) for AtCHK2. For each of constructs, homozygous plant lines were selected with a moderate to strong expression level to be tested for their regeneration capacity.

### Example 5: Regeneration Assays of Arabidopsis Lines Expressing CHK Genes

The regeneration assays used are based on the shoot initiation assays described by To et al. (Plant Cell 16: 658-671, 2004, which incorporated herein by reference). Arabidopsis seedlings of the tested lines were grown on 0.5 MS10 culture medium in square plates placed vertically in the dark for 3-4 days and then in dim light for 3 days to produce elongated and firm hypocotyls. Hypocotyls of around 7 mm in length were excised from the seedlings. Hypocotyl explants were transferred to MS medium (Murashige and Skoog, 1962, Physiol. Plant. 15: 473-497) with 1% (w/v) sucrose and 0.4% (w/v) phytagel containing kinetin (300 ng·mL⁻¹) and NAA (100 ng·mL⁻¹) and kept for 7 weeks at 25° C. at 16 h light/8 h dark. For each transgenic line 10 individual explants were tested in single square plates that also included five non-transformed (negative) control explants of Arabidopsis Col-0 and five explants of the Arabidopsis arr3,4,5,6,8,9 hextuple mutant serving as a positive control for regeneration. This mutant is known to regenerate at high efficiencies under these conditions (To et al., 2004, Plant Cell 16:658-671). In each experiment four independent plates were assayed and the experiment was conducted in duplicate. Shoot regeneration rates were determined based on the number of inflorescence shoot that were formed after 7 weeks of growth.

Regeneration experiments were monitored, imaged and scored over seven weeks. All transgenic lines displayed enhanced regeneration in comparison to the control Col-0 line (Table 2). Regeneration efficiency was expressed as the percentage of explants displaying inflorescence shoot formation out of the total number of explants cultured. It is clear from Table 2 that the regeneration efficiency of plant material expressing CHK2 or CHK4 genes under control of their native promoters outperforms the regeneration efficiency of the positive control (hextuple arr mutant). Overall it is clear that enhanced expression of either CHK2 or CHK4 genes improves the regeneration capacity.

The percentage amino acid identity between CHK2, CHK3 and CHK4 is indicated in Table 3.

TABLE 1A
Sequences

| SEQ ID NO: | Sequence |
|---|---|
| 1 | Arabidopsis thaliana Nucleotide sequence CHK2 |
| 2 | Arabidopsis thaliana Nucleotide sequence CHK3 |
| 3 | Arabidopsis thaliana Nucleotide sequence CHK4 |
| 4 | Arabidopsis thaliana Amino acid sequence CHK2 |
| 5 | Arabidopsis thaliana Amino acid sequence CHK3 |
| 6 | Arabidopsis thaliana Amino acid sequence CHK4 |
| 7 | Arabidopsis thaliana Genomic sequence CHK2 |
| 8 | Arabidopsis thaliana Genomic sequence CHK3 |
| 9 | Arabidopsis thaliana Genomic sequence CHK4 |
| 10 | Arabidopsis thaliana promoter sequence CHK2 |
| 11 | Arabidopsis thaliana promoter sequence CHK3 |
| 12 | Arabidopsis thaliana promoter sequence CHK4 |
| 13 | Arabidopsis thaliana promoter sequence CHK2 with SNP |

TABLE 1B
primer sequences

| SEQ ID NO: | Primer name | Sequence |
|---|---|---|
| 14 | pAtCHK2_F | TAATTTGAATATTTATATTCAATTTCATACATAT |
| 15 | pAtCHK2_R | TTCGACTCCTAATCTCAGATTCA |
| 16 | pAtCHK4_F | CCAATTCACGTTAAATCTATCTCTTG |
| 17 | pAtCHK4_R | CACTTCAAATGTAGGTATTCCATTTT |
| 18 | AtCHK2_F_CDS | ATGTCTATAACTTGTGAGCTCTTGAA |
| 19 | AtCHK2_R_CDS | TTAACAAGGTTCAAAGAATCTTGC |
| 20 | AtCHK2_F_CDS_attB1F | GGGGACAAGTTTGTACAAAAAAGCAGGCTATGTCTATA |
| | | ACTTGTGAGCTCTTGAA |
| 21 | AtCHK2_R_CDS_attB2R | GGGGACCACTTTGTACAAGAAAGCTGGGTTTAACAAG |
| | | GTTCAAAGAATCTTGC |
| 22 | AtCHK4_F_CDS | ATGAGAAGAGATTTTGTGTATAATAATAATGC |
| 23 | AtCHK4_R_CDS | TTACGACGAAGGTGAGATAGGA |
| 24 | AtCHK4_F_CDS_attB1F | GGGGACAAGTTTGTACAAAAAAGCAGGCTATGAGAAG |
| | | AGATTTTGTGTATAATAATAATGC |
| 25 | AtCHK4_R_CDS_attB2R | GGGGACCACTTTGTACAAGAAAGCTGGGTTTACGACG |
| | | AAGGTGAGATAGGA |
| 26 | Q_CHK4_F | TAAGCCTGTTAGAGCTGCTGTG |
| 27 | Q_CHK4_R | TCTTTCAAACGCAGCAGCTG |
| 28 | Q_CHK2_F | GCTACAGAAGAACAGCGTGTTG |
| 29 | Q_CHK2_R | TGTTTGCTGGCAAGTTGGTG |

TABLE 2
Regeneration efficiencies of Arabidopsis hypocotyl explants expressing
CHK alleles on medium with kinetin and NAA

| Type of CHK alleles | # explants | # regener. | % regener. |
|---|---|---|---|
| Effect of CHK alleles | | | |
| AtCHK2 (pKG9785) | 79 | 40 | 50.6 |
| AtCHK4 (pKG9791) | 68 | 43 | 63.2 |
| Controls | | | |
| Arabidopsis arr3, 4, 5, 6, 8, 9 | 360 | 160 | 44.4 |
| hextuple mutant | | | |
| Col-0 negative control | 360 | 7 | 1.9 |

TABLE 3
Percentage amino sequence identity between CHK2, CHK3 and CHK4

| | CHK2 | CHK3 | CHK4 |
|---|---|---|---|
| CHK2 | 100% | 58% | 54% |
| CHK3 | | 100% | 53% |
| CHK4 | | | 100% |

### Example 6: Identifying the Tomato CHK2 Orthologue

An orthology search was carried out with a query of the AtCHK2 and AtCHK4 genes (AT5G35750 and AT2G0183) in 15 plant proteome databases using multiple protein sequence alignment software JackHMMER (http://hmmer.org, version 3.2.1; June 2018; Johnson et al., BMC Bioinformatics, 11: 431, 2010, doi: 10.1186/1471-2105-11-431). JackHMMER blast searches were supplemented with a MEME Suite motif-based sequence analysis (http://meme-suite.org/index.html; Meme Suite 4.12.0., June 2017; Bailey et al., Nucl. Acids Res. 37: W2302-W208, 2009, doi.org/10.1093/nar/gkp335) to identify and visualize known protein domains. The orthology search revealed single candidate genes in the tomato genome for both AtCHK2 and AtCHK4 genes as queries, with amino acid identities of >50%. These tomato genes are Solyc07g047770 (SlyCHK2) and Solyc07g008110 (SlyCHK4) as most likely homologs of AtCHK2 and AtCHK4, respectively. Solyc07g047770 (SlyCHK2) was selected for further work.

### Example 7: Construction of a Tomato CHK Expression Cassette

A functional expression cassette of SlyCHK2 containing 2 kb of its native promoter sequence was designed in silico. The nopaline synthase terminator and Gateway adapters were appended to the sequence to create a cloning fragment for direct cloning in a Gateway destination vector. The synthesis of the cassette was ordered from GeneART (www.thermofisher.com). The full sequence of the cassette is given in SEQ ID NO: 32. The fragment was introduced in Gateway binary destination vector pKm43GW (Karimi et al., Plant Physiol. 145: 1144-1154, 2007, doi.org/10.1104/pp.107.106989) through a single step LR cloning reaction (www.thermofisher.com). pKm43GW is a Gateway multisite binary destination vector containing streptomycin and spectinomycin resistance markers for bacterial selection, and an nptII gene for selection of plant tissue on kanamycin. The resulting plasmid construct was named pKG10867 and was cloned in E. coli and checked by restriction enzyme digestion using the combination EcoRV and NheI. Miniprep plasmid DNA was electroporated to Agrobacterium tumefaciens strain GV3101.

### Example 8: Tomato Transformation

The SlyCHK2 under its native promoter was introduced into tomato cultivar Moneyberg-Plus (TMV-resistant) by Agrobacterium-mediated transformation. Approximately 50 tomato seeds were sterilized and germinated on ½MS10 medium for 11 days. Cotyledon explants from the seedlings were dissected and precultured for 24 h on 2N1B medium (=MS20 medium containing 2 mg·l⁻¹ NAA and 1 mg·l⁻¹ BAP) supplemented with 40 μg·l⁻¹ acetosyringone. The explants were submerged in a suspension of Agrobacterium tumefaciens GV3101 carrying pKG10867 grown overnight in TY medium containing 20 mg·l⁻¹ streptomycin and 50 mg·l⁻¹ spectinomycin, and diluted to OD₆₀₀ 0.138. The explants were blotted dry and cocultivated for 2 days on 2N1B plates with 40 μg·l⁻¹ acetosyringone. Subsequently, the explants were transferred to selective medium MS20ZVCK consisting of MS20 medium with 1 mg·l⁻¹ zeatin, 200 mg·l⁻¹ vancomycin, 200 mg·l⁻¹ cefotaxim and 100 mg·l⁻¹ kanamycin and cultivated at 25° C. and 3000 lux (16/8 h photoperiod) in a growth chamber. The explants were subcultured every 3 weeks onto fresh medium. When callus had formed on the selective medium, the callus was subcultered and the original explants were discarded.

### Example 9: Recording Transformation Efficiencies

Tomato callus subcultured every 3 weeks onto fresh medium MS20ZCVK started to produce shoot meristems and shoots from day 114 of the experiment. Shoots over 5 mm in length were taken off the calli, and transferred to rooting medium consisting of MS20 without any additions. The accumulated number of shoots harvested in this way was recorded (Table 4) and compared to similarly cultivated tomato Moneyberg cotyledon explants that had not been contacted with Agrobacterium tumefaciens and were grown on medium without kanamycin. It is clear that shoots harvested from kanamycin-resistant callus after pKG10867-transformation appear in larger numbers and faster than shoots regenerating from control tomato explants. This effect is attributed to the ectopic expression of the SlyCHK2 gene.

TABLE 4
Shoot regeneration efficiencies of tomato Moneyberg cotyledon explants
transformed with SlyCHK2 (pKG10867), recorded as the cumulative
number of shoots harvested from transformed calli over time.

| | # | day 114 | day 136 | day 156 | day 183 |
|---|---|---|---|---|---|
| Moneyberg pKG10867 | 200 | 1 | 26 | 47 | 93 |
| Moneyberg control | 200 | 1 | 7 | 29 | 64 |

1. A method for improving cytokinin-induced de novo shoot formation of a plant cell, comprising:
(a) increasing or introducing expression of at least one a histidine kinase in the plant cell;
(b) inducing de novo formation of a number of shoots and elongation of the number of shoots by exposing the plant cell of (a) to a cytokinin-comprising medium;
(c) dissecting a de novo formed shoot; and
(d) inducing root formation of the dissected shoot, wherein the number of de novo formed shoots is increased as compared to an identical cell not having an increased or introduced expression of the histidine kinase, and wherein the histidine kinase has at least 80% sequence identity with SEQ ID NO: 4.

2. The method according to claim 1, wherein the histidine kinase is encoded by a nucleotide sequence having at least 50% sequence identity with SEQ ID NO: 1.

3. The method according to claim 1, wherein the expression of the histidine kinase is transiently increased or introduced into the plant cell.

4. The method according to claim 1, wherein the expression of the histidine kinase is continuously increased or introduced in the plant cell.

5. The method according to claim 1, wherein the plant cell is from a plant selected from the group consisting of barley, cabbage, canola, cassava, cauliflower, chicory, cotton, cucumber, eggplant, grape, hot pepper, lettuce, maize, melon, oilseed rape, potato, pumpkin, rice, rye, sorghum, squash, sugar cane, sugar beet, sunflower, sweet pepper, tomato, water melon, wheat, zucchini, soybean, chrysanthemum and Arabidopsis.

6. The method according to claim 1, wherein the cytokinin is an adenine-type cytokinin.

7. The method according to claim 6, wherein the adenine-type cytokinin is selected from the group consisting of kinetin, zeatin, trans-zeatin, cis-zeatin, dihydrozeatin, 6-benzylaminopurine and 2iP.

8. The method according to claim 1, further comprising:
(a) incubating the plant cell having increased or introduced expression of the histidine kinase in the medium comprising a cytokinin plant hormone; and
(b) allowing the plant cell to regenerate into a plant.

9. The method according to claim 3, wherein the medium comprises at least one further plant hormone.

10. The method according to claim 9, wherein the one further plant hormone is an auxin.

11. The method according to claim 8, wherein the plant cell is part of at least one of a multicellular tissue, a callus tissue, a plant organ, an explant, a hypocotyl explant, a stem explant, a cotyledon explant, a root explant, a leaf explant, a flower explant and a meristematic tissue.

12. A method according to claim 8, wherein the concentration of cytokinin in the medium is 100-3000 ng/ml.

13. The method according to claim 12, wherein the concentration of cytokinin in the medium is 200-600 ng/ml.

14. The method according to claim 1, wherein the plant cell is incubated in a medium comprising a cytokinin for at least 5 weeks prior to dissecting a de novo formed shoot.

15. The method according to claim 1, wherein the plant cell is a plant cell of a recalcitrant plant.

16. The method according to claim 1, wherein the amino acid sequence of the histidine kinase has at least 90% sequence identity with SEQ ID NO: 4.

17. The method according to claim 1, wherein the amino acid sequence of the histidine kinase has at least 95% sequence identity with SEQ ID NO: 4.
